(19) United States
(12) Patent Application Publication
(10) Pub. No.: US 2022/0127569 A1
(43) Pub. Date: Apr. 28, 2022

(54) METHODS OF PROMOTING THYMIC EPITHELIAL CELL AND THYMIC EPITHELIAL CELL PROGENITOR DIFFERENTIATION OF PLURIPOTENT STEM CELLS, RESULTING CELLS, AND USES THEREOF

(71) Applicant: THE TRUSTEES OF COLUMBIA UNIVERSITY IN THE CITY OF NEW YORK, New York, NY (US)

(72) Inventors: Rafael Gras Pena, New York, NY (US); Megan Sykes, New York, NY (US); Nichole Danzi, New York, NY (US); Mohsen Khosravi-Maharlooei, New York, NY (US)

(21) Appl. No.: 17/492,137

(22) Filed: Jan. 12, 2022

Related U.S. Application Data
(63) Continuation of application No. PCT/US2020/025955, filed on Mar. 31, 2020.
(60) Provisional application No. 62/827,383, filed on Apr. 1, 2019.

(51) Int. Cl. C12N 5/078; A61P 37/00; A61K 35/26

(52) U.S. Cl. CPC C12N 5/0634; A61P 37/00; A61K 35/26; C12N 2501/155; C12N 2501/15; C12N 2501/16; C12N 2501/41; C12N 2501/115; C12N 2506/02

(57) ABSTRACT

The current disclosure provides for methods of promoting differentiation of pluripotent stem cells into thymic epithelial cells or thymic epithelial cell progenitors as well as the cells obtained from the methods, and solutions, compositions, and pharmaceutical compositions comprising such cells. The current disclosure also provides for methods of using the thymic epithelial cells or thymic epithelial cell progenitors for treatment and prevention of disease, generating organs, as well as other uses, and kits.

### CROSS REFERENCE TO RELATED APPLICATION

The present application is a continuation of PCT Application No. PCT/US2020/025955, filed Mar. 31, 2020, which claims priority to U.S. patent application Ser. No. 62/827,383 filed Apr. 1, 2019, all of which are incorporated by reference, as if expressly set forth in their respective entireties herein.

### STATEMENT OF GOVERNMENT SUPPORT

This invention was made with government support under grant numbers DK104207, DK103585 and AI045897, awarded by the National Institutes of Health. The government has certain rights in this invention.

### FIELD

The current disclosure provides for methods of promoting differentiation of pluripotent stem cells into thymic epithelial cells or thymic epithelial cell progenitors as well as the cells obtained from the methods, and solutions, compositions, and pharmaceutical compositions comprising such cells. The current disclosure also provides for methods of using the thymic epithelial cells or thymic epithelial cell progenitors for treatment and prevention of disease, generating organs, as well as other uses, and kits.

### BACKGROUND

The thymus is the primary lymphoid organ responsible for T cell development and education. Thymic epithelial cells (TECs) are a key component of the thymic stroma. TECs in the thymic cortex (cTECs) are specialized for T cell positive selection, while medullary TECs (mTECs) are involved in T cell negative selection. TEC-mediated selection promotes a self-tolerant and highly diverse T cell repertoire that can recognize foreign antigens presented by self-MHC molecules. Normal thymopoiesis involves a highly organized network of stromal and hematopoietic cell types in addition to TECs.

In vitro generation of functional TECs or TECs progenitors (TEPs) from human pluripotent stem cells (hPSCs) could generate cells, tissues or organs which aid in T cell reconstitution in patients with thymic dysfunction due to congenital disorders such as DiGeorge syndrome and acquired dysfunction due to HIV infection, high dose chemotherapy and radiotherapy treatment, graft-vs-host disease and long-term immunosuppressive therapy combined with advanced age, which in itself results in poor thymopoietic function. As the number of TECs in human adult thymi is limited and reliable methods of expanding them from post-natal thymi have been elusive, generating TECs from pluripotent stem cells (PSCs) is an important goal. Creating an in vitro protocol for tightly controlled differentiation of hPSCs to TECs requires precise knowledge and application of developmental temporal and cytokine cues. While the generation of functional TEPs from murine or human PSCs that support murine (Parent et al. 2013; Sun et al. 2013; Soh et al. 2014; Bredenkamp et al. 2014)) or human (Su et al. 2015) T cell development has been described, reconstitution of high levels of naïve human T cells has not been demonstrated. Thus, there is a need in the art for a method to generate human TEPs and TECs.

### SUMMARY

Shown herein is an efficient method to induce differentiation of human pluripotent stem cells (hPSCs) including embryonic stem cells (ESCs) and induced pluripotent stem cells (iPSCs) into thymic epithelial cell progenitors (TEC progenitors) in vitro, wherein the thymic epithelial cells (TECs) or thymic epithelial cell progenitors (TEPs) are capable of generating thymic organs and T cells in vivo.

This protocol achieved the highest in vitro expression of FOXN1 described so far without protein transduction or genetic modification. After culture, the cells expressed epithelial markers EpCam, Keratin 5 and Keratin 8. When mixed with human thymic mesenchymal cells (ThyMES), the cells implanted in vivo supported naïve human T cell reconstitution in thymectomized NOD-scid IL2Rgammanu^{null} (NSG) mice (Khosravi-Mahrarlooei et al. 2020) receiving human hematopoietic stem cells (HSCs) intravenously.

One embodiment of the present disclosure is a method of inducing differentiation of human pluripotent stem cells (hPSCs) including embryonic stem cells (ESCs) and induced pluripotent stem cells (iPSCs) into thymic epithelial cells (TECs) or thymic epithelial cell progenitors (TEC progenitors) (TEPs) including the steps of:

1. differentiating human pluripotent stem cells into endoderm cells;
2. culturing the resulting endoderm cells and differentiating the endoderm cells into anterior foregut cells by contacting or incubating the endoderm cells with an agent which inhibits BMP and an agent which inhibits TGFβ signaling, and further contacting or incubating the cells with an agent which stimulates the expression of HOXA3 and an agent which stimulates the expression of TBX1;
3. further culturing the resulting anterior foregut cells and differentiating the anterior foregut cells into pharyngeal endoderm cells by contacting or incubating the anterior foregut cells with an agent which stimulates the expression of TBX1 and an agent which stimulates the expression of PAX9 and PAX1;
4. further culturing the resulting pharyngeal endoderm cells and differentiating the pharyngeal endoderm cells into distal pharyngeal pouch (PP) specification cells, thymic epithelial cells or thymic epithelial cell progenitors by contacting or incubating the pharyngeal endoderm cells with an agent which inhibits BMP and subsequently contacting or incubating the pharyngeal endoderm cells with BMP; and
5. contacting or incubating the TECs or TEPs at the end of the method with a survivin inhibitor.

A further embodiment is a method of obtaining thymic epithelial cells (TECs) or thymic epithelial cell progenitors (TEPs) from human pluripotent stem cells (hPSCs) including embryonic stem cells (ESCs) and induced pluripotent stem cells (iPSCs) including the steps of:

1. differentiating human pluripotent stem cells into endoderm cells;
2. culturing the resulting endoderm cells and differentiating the endoderm cells into anterior foregut cells by contacting or incubating the endoderm cells with an agent which inhibits BMP and an agent which inhibits TGFβ signaling, and further contacting or incubating the cells with an agent which stimulates the expression of HOXA3 and an agent which stimulates the expression of TBX1;
3. further culturing the resulting anterior foregut cells and differentiating the anterior foregut cells into pharyngeal endoderm cells by contacting or incubating the anterior foregut cells with an agent which stimulates the expression of TBX1 and an agent which stimulates the expression of PAX9 and PAX1;
4. further culturing the resulting pharyngeal endoderm cells and differentiating the pharyngeal endoderm cells into distal pharyngeal pouch (PP) specification cells, thymic epithelial cells by contacting or incubating the pharyngeal endoderm cells with an agent which inhibits BMP and subsequently contacting or incubating the pharyngeal endoderm cells with BMP; and
5. contacting or incubating the TECs or TEPs at the end of the method with a survivin inhibitor.

A further embodiment of the present disclosure is a method of inducing differentiation of human pluripotent stem cells (hPSCs) including embryonic stem cells (ESCs) and induced pluripotent stem cells (iPSCs) into thymic epithelial cells (TECs) or thymic epithelial cell progenitors (TEC progenitors) (TEPs) including the steps of:

1. differentiating the pluripotent stem cells into endoderm cells by culturing the pluripotent stem cells in serum-free differentiation medium and contacting or incubating the cells with human Bone Morphogenic Protein (BMP), human b Fibroblast Growth Factor (bFGF) and human Activin A;
2. differentiating the endoderm cells from the first step into anterior foregut cells by culturing the endoderm cells in differentiation medium and contacting or incubating the cells with Noggin, SB431542, retinoic scid and FGF8b;
3. differentiating the anterior foregut cells from the second step into pharyngeal endoderm cells by culturing the cells in differentiation medium, and contacting or incubating the cells with FGF8b and retinoic acid followed by FGF8b and Sonic Hedgehog (Shh);
4. differentiating the pharyngeal endoderm cells from step 3 into 3rd pharyngeal pouch specification by culturing the cells in differentiation medium and contacting or incubating the cells with Noggin;
5. further differentiating the pharyngeal endoderm cells from step 3 or step 4 into 3rd pharyngeal pouch specification cells, TEPs or TECs, by culturing the cells in differentiation medium and contacting or incubating the cells with BMP; and
6. exposing the cells to a survivin inhibitor.

A further embodiment is a method of obtaining thymic epithelial cells (TECs) or thymic epithelial cell progenitors (TEPs) from human pluripotent stem cells (hPSCs) including embryonic stem cells (ESCs) and induced pluripotent stem cells (iPSCs) including the steps of:

1. differentiating the pluripotent stem cells into endoderm cells by culturing the pluripotent stem cells in serum-free differentiation medium and contacting or incubating the cells with human Bone Morphogenic Protein (BMP), human b Fibroblast Growth Factor (bFGF) and human Activin A;
2. differentiating the endoderm cells from the first step into anterior foregut cells by culturing the endoderm cells in differentiation medium and contacting or incubating the cells with Noggin, SB431542, retinoic acid and FGF8b;
3. differentiating the anterior foregut cells from the second step into pharyngeal endoderm cells by culturing the cells in differentiation medium, and contacting or incubating the cells with FGF8b and retinoic acid followed by FGF8b and Sonic Hedgehog (Shh);
4. differentiating the pharyngeal endoderm cells from step 3 into 3rd pharyngeal pouch specification by culturing the cells in differentiation medium and contacting or incubating the cells with Noggin;
5. further differentiating the pharyngeal endoderm cells from step 3 or step 4 into 3rd pharyngeal pouch specification cells, TEPs or TECs, by culturing the cells in differentiation medium and contacting or incubating the cells with BMP; and
6. exposing the cells to a surviving inhibitor.

In some embodiments, the contacting or incubating of the cells with the various agents is accomplished by culturing the cells in media comprising the agents.

The current disclosure also provides for cells obtained using the methods described herein, and solutions, compositions, and pharmaceutical compositions comprising the cells obtained using the methods described herein.

In some embodiments, these cells express FOXN1, EpCAM, Keratin 5, and Keratin 8. In some embodiments, these cells are thymic epithelial cells (TECs). In some embodiments, these cells are thymic epithelial cell progenitors (TEC progenitors) (TEPs).

All of the foregoing embodiments including cells, solutions, compositions, and pharmaceutical compositions comprising the cells can be used to treat and/or prevent disease.

In some embodiments, the disease is a disease of the thymus.

In further embodiments, the disease is an autoimmune disease, including but not limited to Type 1 diabetes, rheumatoid arthritis (RA), psoriasis, psoriatic arthritis, multiple sclerosis, systemic lupus erythematosus (SLE), inflammatory bowel disease, Addison's disease, Graves' disease, Sjögren's syndrome, Hashimoto's thyroiditis, myasthenia gravis, autoimmune vasculitis, pernicious anemia, celiac disease, vitiligo and alopecia areata.

All of the foregoing embodiments including cells, solutions, compositions, and pharmaceutical compositions comprising the cells can be used to recover or restore impairment of the function of the thymus wherein the impaired functionality is due to aging or injury or infectious diseases such as HIV.

All of the foregoing embodiments including cells, solutions, compositions, and pharmaceutical compositions comprising the cells can be used to reconstitute T cells after a bone marrow transplant.

All of the foregoing embodiments including cells, solutions, compositions, and pharmaceutical compositions comprising the cells can be used to generate a hybrid thymus comprising the cells and a thymus or other cells or tissues which comprise a thymus. In some embodiments, the thymus is from a different individual. In some embodiments, the thymus is from a different species. In some embodiments, the thymus is from a swine. In some embodiments, the swine is a fetal swine. In some embodiments, the swine is a juvenile swine.

All of the foregoing embodiments including cells, solutions, compositions, and pharmaceutical compositions comprising the cells can be used to develop mouse models and perform drug testing.

All of the foregoing embodiments including cells, solutions, compositions, and pharmaceutical compositions comprising the cells can be used to develop a thymus for the treatment of individuals with congenital abnormalities, where the thymus function is partially or totally impaired, like DiGeorge Syndrome, 22q.11.2 deletion syndrome or nude syndrome.

In yet additional embodiments, the disclosure relates to kits for practicing the methods of the disclosure to obtain cells, solutions, compositions, and pharmaceutical compositions disclosed herein. The disclosure also includes kits comprising the cells, solutions, compositions, and pharmaceutical compositions.

As described herein, the methods, systems and kits are suitable for the large-scale, reproducible production of thymic epithelial cells or thymic epithelial cell progenitors (TEPs).

### BRIEF DESCRIPTION OF THE FIGURES

For the purpose of illustrating the invention, there are depicted in drawings certain embodiments of the invention. However, the invention is not limited to the precise arrangements and instrumentalities of the embodiments depicted in the drawings.

FIG. 1—Establishment of a protocol for direct differentiation of hESCs to 3rd PP biased Pharyngeal Endoderm. FIG. 1A is a schematic of the representation of postulated hESC differentiation steps towards desired cell-fates, mirroring the aims of the treatments shown in FIG. 1B. FIG. 1B is a schematic of the tested protocols for hESCs differentiation to 3rd PP biased pharyngeal endoderm until day 15. Protocol #1 (indicated as “1” in FIG. 1B) (FGF8b+RA₂₅₀) was considered the reference protocol to which protocol #2 (indicated as “2” in FIG. 1B) (FGF8b) (#1 vs #2) and #3 (indicated as “3” in FIG. 1B) (FGF8b+RA250 to FGF8b+Shh) (#1 vs #3), are compared in FIG. 1D. In FIG. 1B, “NS” indicates Noggin and SB431542. FIG. 1C shows representative flow cytometric analysis of EpCAM and CXCR4 (endodermal markers) expression on dissociated embryoid bodies at day 4.5. FIG. 1D is a graph showing the comparative analyses of gene expression in differentiated hESCs at day 15 under protocol conditions shown in FIG. 1B. The graphs represent fold change in RNA expression as measured by qPCR. (n=3-11, values represent mean±SEM, *p<0.05, **p<0.01, ***p<0.001, two-tailed ratio paired t-test). FIG. 1E shows the comparison of PP markers' expression at day 15 in hESCs differentiated using protocol #1 with hESCs differentiated to ‘liver’ (‘hepatic conditions’ (Gouon-Evans et al. 2006)). Bar graphs represent fold change in RNA expression as measured by qPCR (n=6, values represent mean+SEM, *p<0.05, **p<0.01, ***p<0.001, two-tailed ratio paired t-test). FIG. 1F is a graph showing the comparative analyses of gene expression in differentiated hESCs at day 15 under protocol conditions shown in FIG. 1B. The graphs represent fold change in RNA expression as measured by qPCR. (n=9-11, values represent mean±SEM, *p<0.05, **p<0.01, ***p<0.001, two-tailed ratio paired t-test).

FIG. 2—Development of a protocol for distalization of 3rd PP and/or TEC. FIG. 2A is a schematic of the tested protocols for distalization of 3rd PP biased cells until day 30. In FIG. 2A “3b” and “3c” indicate modifications based on Protocol #3 in FIG. 1B; “4b” and “4c” indicate modifications based on Protocol #4 in FIG. 1B. FIG. 2B shows a schematic representation of multiple hESC differentiation protocols tested under divergent culture conditions from day 6.5 onwards. hESCs were differentiated to definitive endoderm (DE) for 4.5 days and subsequently anteriorized with Noggin+SB (NS) and retinoic acid (RA). Then the cells were patterned for 8.5 days with RA and different combinations of the indicated factors, until day 15. FIG. 2C are graphs of expression analysis of FOXA2, HOXA3, SIX1, TBX1, EYA1, PAX9 and PAX1 in the hESC-derived cells from cultures containing RA and FGF8b (protocol #1) vs RA+factors substituting FGF8b as shown in FIG. 2B. Bar graphs represent fold change in RNA expression as measured by qPCR (n=3, values represent mean±SEM, *p<0.05, **p<0.01, ***p<0.001, one-way ANOVA with Dunnett's multiple comparisons test). FIG. 2D show the effect of Noggin exposure on PAX9 expression at day 30. The bar graphs represent fold change in PAX9 expression between protocol #3b vs #3c and #4b vs #4c. (n=4, values represent mean±SEM, *p<0.05, **p<0.01, ***p<0.001, two-tailed ratio paired t-test). FIG. 2E shows the fold change of FOXN1 expression at day 30 upon initiation of FGF8b treatment at day 4.5 vs day 6.5 (protocol #3c vs #4c) as measured by qPCR (n=4-8, values represent mean±SEM, *p<0.05, **p<0.01, ***p<0.001, two-tailed ratio paired t-test). FIG. 2F shows the fold change in FOXN1 expression at day 21 vs day 30 (before and after BMP4 exposure) of protocol #4c as measured by qPCR (n=4-8, values represent mean±SEM, *p<0.05, **p<0.01, ***p<0.001, two-tailed ratio paired t-test). FIG. 2G shows the fold change in FOXN1 expression at day 15 vs day 30 of protocol #4c as measured by qPCR (n=4-8, values represent mean±SEM, *p<0.05, **p<0.01, ***p<0.001, two-tailed ratio paired t-test).

FIGS. 3A-3C—Characterization of in vitro differentiated TEC progenitors at day 30. FIG. 3A shows the TEC marker expression in cultured cells (d30; protocol #4c) compared to fetal thymus (FTHY). (Ct relative to β-actin, n=3-22, values represent mean+SEM, *p<0.05, **p<0.01, ***p<0.001, two-tailed unpaired Welch's t-test). Every dot represents an independent experiment. FIG. 3B shows the 3rd PP marker expression in H9 cells cultured under protocol #4c conditions for 30 days compared to fetal thymus. Bar graphs represent mean Ct values relative to β-actin+SEM (n=3-6). Two-tailed unpaired Welch's t-test. Each dot represents an independent experiment. FIG. 3C are graphs of Pearson correlation analysis of gene expression levels of FOXN1 and GCM2, FOXN1 and IL7, and FOXN1 and CD205. Both axes depict Ct values relative to β-actin. Every dot represents an independent experiment.

FIG. 4—Treatment of day 30 hES-TEP cultures with survivin inhibitor YM155 depletes multipotent cells. FIG. 4A is a schematic representation of protocol #4c showing time period of YM155 treatment. This schematic also shows the complete differentiation protocol. FIG. 4B is a graph of Pearson correlation analysis of FOXN1 and OCT4 expression. Both axes depict Ct values relative to β-actin. Every dot represents an independent experiment. FIG. 4C is a graph of the fold change in OCT4 expression at day 30 following depletion of multipotent cells (protocol #4c vs #4c+YM155; n=5, values represent mean+SEM, *p<0.05, two-tailed ratio paired t-test). FIG. 4D is a graph showing percent survival free from overt teratoma formation in weeks post hES-TEP transplantation. hES-TEP-grafted mice from protocol #4c day 15 (n=8, grey line), compared to hES-TEP-grafted mice from day 30 cultures treated with (n=15, black dotted line) or without (n=12, solid black line) YM155. Log-rank Mantel Cox test showed p<0.005 for hES-TEP day 15 survival compared to either hES-TEP day 30 alone or hES-TEP day 30+YM155 treatment.

FIG. 5—Reaggregate hES-TEP prepared using the protocol shown in FIG. 4A and thymic mesenchyme cells form a thymic organoid that supports thymopoiesis. FIG. 5A shows the percentage of T cells when the native thymic rudiment was surgically removed (ATX) or not from NSG mice injected with human HSCs. ACK lysis of peripheral blood produced white blood cells (WBCs) that were stained for HuCD45+CD3+ T cells at the indicated weeks post-HSC injection. NSG n=12, ATX NSG n=4. FIG. 5B are representative FACS plots gated on HuCD45+CD19−CD14− cells. NSG n=10, ATX n=14. FIGS. 5C-5F show the frequency of various cells when cultured hES-TEPs clusters mixed with thymic mesenchyme cells (TMC) or TMCs alone were grafted under the renal capsule of ATX NSG mice injected with human HSCs. FIG. 5C shows the frequency of HuCD45+ cells among total mouse+human CD45+ cells in PBMCs for individual hES-TEC/TMC mice and the average (grey line) for TMC grafted mice (n=6). FIG. 5D shows the frequency of CD3+ cells among total mouse+human CD45+ cells in PBMCs for individual hES-TEP/TMC mice and the average (grey line) for TMC grafted mice (n=6). FIG. 5E shows the frequency of CD4+ cells among total mouse+human CD45+ cells in PBMCs for individual hES-TEP/TMC mice and the average (grey line) for TMC grafted mice (n=6). FIG. 5F shows the frequency of CD4+ cells stained for CD45RA+CD45RO− naïve cells. Timepoints with fewer than 100 CD4+ events were excluded. FIG. 5G show human T cells in PBMCs from a healthy human (left), hES-TEC/TMC (middle) and TMC mouse (right) 30 weeks post-humanization. hES-TEC/TMC plot is representative of n=4 mice that developed CD4+ and CD8+ T cells and TMC plot is representative of n=6. FIG. 5H shows the CD4+ and CD8+ expression on cells from hES-TEC/TMC (n=3). Cell suspensions were gated on HuCD45+CD19− CD14− cells.

FIG. 6—hES-TECs generated from TEPs prepared using the protocol shown in FIG. 4A persist in swine thymus and promote thymopoiesis. FIG. 6A is schematic of the protocol to test the hES-TECs in vivo. The swine thymus was injected or not with hES-TEPs and grafted under the renal capsule of ATX NSG mice injected i.v. with human HSCs. FIG. 6B shows the results of flow cytometry analysis of the thymic grafts 18-22 weeks post-transplant. Single cell suspension from liberase digested stromal fraction of half the thymus graft was stained and analyzed by flow cytometry. Human pediatric thymus was prepared as a control. Non-hematopoietic cells were gated as huCD45-HLA-ABC+. Markers for thymic fibroblasts (CD105+) and epithelial cell marker EpCAM are shown. FIG. 6C is a graph of the frequency of huCD45-HLA-ABC+CD105-EpCAM+ epithelial cells in SwTHY+hES-TECs (left bar, squares) and SwTHY (right bar, triangles) grafts. FIG. 6D are representative flow cytometry plots of thymocytes gated as huCD45+CD19−CD14− cells for CD4/CD8 distribution for human pediatric thymus, and swine thymus injected or not injected with hES-TEPs (left to right). FIG. 6E are graphs of absolute count of thymocytes from half of the thymus graft in double positive CD4+CD8+, single positive CD4+CD8- and CD4-CD8+ with further division into immature CD45RO+ compared to more mature CD45RA+ thymocytes are shown. Average+SEM are shown for SwTHY+hES-TEC (n=6, squares) and SwTHY (n=5, triangles) from two independent experiments. Thymic grafts yielding fewer than 6×10⁵ (n=1 each from SwTHY+hES-TEC and SwTHY) cells were eliminated from analysis. Mann-Whitney test was used to determine p-values comparing SwTHY+hES-TEC to SwTHY groups with p<0.05 considered significant. +p=0.05, *p<0.05, **p<0.005. FIG. 6F is a graph of human immune cells assayed for total human (huCD45+) cells in PBMCs at the indicated weeks post-humanization. Average+SEM are shown for swine thymus alone (n=9, black line with triangles) and swine thymus injected with hES-TEP (n=11, green line with squares) from two independent hES-TEC differentiations. FIG. 6G is a graph of human immune cells assayed for total B cells (huCD19+) cells in PBMCs at the indicated weeks post-humanization. Average+SEM are shown for swine thymus alone (n=9, black line with black triangles) and swine thymus injected with hES-TEP (n=11, green line with squures) from two independent hES-TEP differentiations. FIG. 6H is a graph of 18-22 weeks post humanization total human CD45+immune cells in the spleen analyzed by flow cytometry. Average+SEM are shown for swine thymus injected with hES-TEP (n=7, squares) and swine thymus alone (n=6, triangles) from two independent hES-TEC differentiations. FIG. 6I is a graph of 18-22 weeks post humanization total human CD19+ B cells in the spleen analyzed by flow cytometry. Average+SEM are shown for swine thymus injected with hES-TEP (n=7, squares) and swine thymus alone (n=6, triangles) from two independent hES-TEP differentiations. FIG. 6J is a graph of 18-22 weeks post humanization total human CD14+ myeloid cells in the spleen analyzed by flow cytometry. Average+SEM are shown for swine thymus injected with hES-TEP (n=7, squares) and swine thymus alone (n=6, triangles) from two independent hES-TEP differentiations.

FIG. 7—hES-TEP prepared using the protocol shown in FIG. 4A injected into swine thymus promotes an increase in the proportion of CD4+ T cells in the blood and increased number of naïve T cells and CD4+ recent thymic emigrants in spleen compared to swine thymus-grafted control mice. FIGS. 7A-7C show the results of human immune cells assayed in PBMCs at the indicated weeks post-humanization. Average+SEM are shown for swine thymus alone (n=9, black line with triangles) and swine thymus injected with hES-TEP (n=11, green line with squares) from two independent hES-TEP differentiations. FIG. 7A shows CD3+ cells. FIG. 7B shows CD8+ cells. FIG. 7C shows CD4+ cells. Significant effect of TEP injection was revealed by two-way ANOVA with p<0.05 considered significant in CD3+ and CD4+ kinetics. Post-hoc Bonferroni multiple comparison at each time point p<0.05 indicated by *. FIG. 7D shows the absolute number of CD3+ T cells in the spleen 18-22 weeks post-humanization. FIG. 7E shows the absolute number of CD8+ T cells in the spleen 18-22 weeks post-humanization. FIG. 7F shows the absolute number of CD4+ T cells in the spleen 18-22 weeks post-humanization. FIG. 7G shows CD45RA versus CCR7 used to distinguish naïve, effector memory (EM), central memory (CM) and terminally differentiation effector memory cells re-expressing CD45RA (EMRA) (left panel) among CD8+(middle panel) or CD4+ T cells (right panel). FIG. 7H shows the absolute number of recent thymic emigrant CD31+CD4+naïve cells as defined CD45RA+CCR7+ cells in mononuclear cells of the spleen. Average+SEM are shown for swine thymus injected with hES-TEP (n=7, squares) and swine thymus alone (n=6, triangles) from two independent hES-TEP differentiations. Mann-Whitney test was used to determine p-values comparing SwTHY alone to SwTHY hES-TEP injected groups with p<0.05 considered significant. *p<0.05.

### DETAILED DESCRIPTION

### Definitions

The terms used in this specification generally have their ordinary meanings in the art, within the context of this invention and the specific context where each term is used. Certain terms are discussed below, or elsewhere in the specification, to provide additional guidance to the practitioner in describing the methods of the invention and how to use them. Moreover, it will be appreciated that the same thing can be said in more than one way. Consequently, alternative language and synonyms may be used for any one or more of the terms discussed herein, nor is any special significance to be placed upon whether or not a term is elaborated or discussed herein. Synonyms for certain terms are provided. A recital of one or more synonyms does not exclude the use of the other synonyms. The use of examples anywhere in the specification, including examples of any terms discussed herein, is illustrative only, and in no way limits the scope and meaning of the invention or any exemplified term. Likewise, the invention is not limited to its preferred embodiments.

As used herein, the term “induced pluripotent stem cells” commonly abbreviated as iPS cells or iPSCs, refers to a type of pluripotent stem cell artificially generated from a non-pluripotent cell, typically an adult somatic cell, or terminally differentiated cell, such as fibroblast, a hematopoietic cell, a myocyte, a neuron, an epidermal cell, or the like.

As used herein, the terms “differentiation” and “cell differentiation” refer to a process by which a less specialized cell (i.e., stem cell) develops or matures or differentiates to possess a more distinct form and/or function into a more specialized cell or differentiated cell, (i.e., thymic epithelial cell).

As used herein, the expressions “cell,” “cell line,” and “cell culture” are used interchangeably and all such designations include progeny. Thus, the words “transformants” and “transformed cells” include the primary subject cell and cultures derived therefrom without regard for the number of transfers. It is also understood that not all progeny will have precisely identical DNA content, due to deliberate or inadvertent mutations. Mutant progeny that have the same function or biological activity as screened for in the originally transformed cell are included.

Where distinct designations are intended, it will be clear from the context.

With respect to cells, the term “isolated” refers to a cell that has been isolated from its natural environment (e.g., from a tissue or subject). The term “cell line” refers to a population of cells capable of continuous or prolonged growth and division in vitro. Often, cell lines are clonal populations derived from a single progenitor cell. It is further known in the art that spontaneous or induced changes can occur in karyotype during storage or transfer of such clonal populations. Therefore, cells derived from the cell line referred to may not be precisely identical to the ancestral cells or cultures, and the cell line referred to includes such variants. As used herein, the terms “recombinant cell” refers to a cell into which an exogenous DNA segment, such as DNA segment that leads to the transcription of a biologically-active polypeptide or production of a biologically active nucleic acid such as an RNA, has been introduced.

### Abbreviations

hPSC—human pluripotent stem cell
ES or ESC—embryonic stem cell
iPSC—induced pluripotent stem cells
TEC—thymic epithelial cell
TEP—thymic epithelial cell progenitor
PE—pharyngeal endoderm
DE—definitive endoderm
AFE—anterior foregut or anterior foregut endoderm
PA—pharyngeal arches
3rd PP—third pharyngeal pouch
Shh—sonic hedgehog
RA—retinoic acid
SP—single positive
DP—double positive

To differentiate DE to third PP, the co-expression of TBX1 and HOXA3 was induced using a combination of FGF8 and retinoic acid (RA). RA treatment was previously shown to boost HOXA3 activity (Parent et al. 2013; Diman et al. 2011), but the TBX1 upregulating potential of FGF8 was a novel finding disclosed herein. It is believed that FGF8 plays a two-fold role in the disclosed differentiation protocol: i) FGF8 signaling immediately after activin exposure drives Tbx1, anteriorizing the DE into a pharyngeally biased AFE (Green et al. 2011). Early exposure to FGF8 (day 4.5 vs day 6.5; protocol #3c vs #4c) strongly pushed the culture towards pharyngeal AFE, significantly increasing the number of FOXN1+ cells at day 30; ii) After anteriorization, FGF8b contributes to development of PE, now acting downstream of and in conjunction with TBX1 (Vitelli et al. 2002; Vitelli et al. 2010).

Another cytokine playing a key role in PE development is sonic hedgehog (Shh) (Moore-Scott and Manley 2005). RA exposure was reduced and replaced with Shh (protocol #1 vs #3) as another innovation. This upregulated PAX9, PAX1, and TBX1, but downregulated HOXA consistent with previous reports showing that Shh signaling induces Tbx1 in PE (Garg et al. 2001). High levels of HOXA3 are critical to early pharyngeal region patterning but its expression diminishes in later stages. Indeed, Pax1 expression is reduced in Hoxa3 null mutants, while Hoxa3 expression is normal in Pax1; Pax9 double mutant embryos (Moore-Scott and Manley 2005). Hoxa3 expression is also unaffected in Shh^{-/-} mutants. Thus, the contribution of temporally opposite gradients of HOXA3 and Pax1-Pax9 to third PP development further justifies the initial use of RA followed by the treatment with Shh in the disclosed protocol.

In the last part of the protocol, the cells were exposed to Noggin and then BMP4. Although it has been shown that BMP signaling is required for FOXN1 expression (Patel et al. 2006; Swann et al. 2017), this is the first report of using Noggin, a BMP4 antagonist and/or inhibitor, for in vitro thymic differentiation. Noggin's presence in the 3^{rd} PP endoderm has been associated with the parathyroid domain rather than the thymus, where BMP4 is expressed (Patel et al. 2006). In the disclosed protocol, adding ectopic Noggin to the culture further enhanced the expression of PAX9 at day 30. Since BMP4 expression starts at E10.5 in cells of the 3rd PP endoderm right after Noggin expression at E9.5 (Patel et al. 2006), the cells were exposed to BMP4 from day 21 to 30 (immediately after Noggin). This led to increases in FOXN1 at day 30 compared to day 21 and day 15. Interestingly, BMP4 treatment without prior exposure to Noggin did not lead to any FOXN1 increase, confirming the need for Noggin exposure to develop sensitivity to BMP4.

Several groups have reported the ability to generate murine and human TEPs from PSCs (Parent et al. 2013; Sun et al. 2013; Soh et al. 2014; Su et al. 2015; Lai and Jim 2009). In three reports, grafts consisting of these cells, often along with supporting mesenchyme or EPCAM− cells from TEP cultures, have reconstituted murine T cells in nude mice, and though robust, continuous thymopoiesis in a normal-appearing thymic structures was not demonstrated. Indeed, the possibility that a wave of thymopoiesis was followed by peripheral lymphopenia-driven expansion of mature T cells was not ruled out. In one report, human T cell repopulation of peripheral tissues and human thymopoiesis in the grafted tissue was demonstrated, though thymic structure was not demonstrated for the grafted cells. Again, peripheral markers of recent thymic emigration were not included in the study, so it is unclear how robust or durable the thymopoiesis was.

Described herein, the hPSC-TEC-dependent appearance of naïve human T cells in the periphery of the mice implanted with hPSC-TEPs plus thymic mesenchymal cells and receiving human HSCs was clearly demonstrated. Since the NSG mouse thymus is also capable of supporting human thymopoiesis, all NSG mice were thymectomized before implanting the hPSC-TEPs (Khosravi et al. 2020), thereby assuring that all peripheral T cells arose from the grafted tissue. The phenotype of peripheral human T cells in these mice eventually converted to the memory type.

The inability to generate durable, structured thymi from “stand-alone” cellular grafts led to the development of a novel approach to assess thymopoietic function of hPSC-TEPs in vivo. It has been previously demonstrated that fetal porcine thymic tissue supports phenotypically normal human thymopoiesis (Nikolic and Sykes 1999) with a diverse TCR repertoire (Shimizu et al. 20008) and robust population of peripheral naïve T cells in NSG mice, though with some subtle differences from that observed for T cells developing in a human thymus graft (Kalscheuer et al. 2014). These fetal pig thymus fragments grow markedly and contain up to hundreds of millions of human thymocytes in a normal-appearing thymic structure (Nikolic and Sykes 1999; Kalscheuer et al. 2014). Disclosed herein is a methodology for injecting hPSC-TEPs into fragments of fetal pig thymus tissue that maintained the human cells in close proximity to the pig thymus tissue and ultimately resulted in their incorporation into the pig thymus as it grew. The human TEPs incorporated into the pig thymus clearly expressed human cTEC and mTEC-associated cytokeratins and appeared integrated into the highly organized thymic structure of the grafts. Most importantly, they had a notable functional effect, significantly increasing the total number of human thymocytes and the number of peripheral naïve human T cells, including CD4+CD34RA+ T cells with the CD31+ RTE phenotype.

Methods and Systems of Obtaining Thymic Epithelial Cells and/or Thymic Epithelial Cell Progenitors

The methods and systems described herein not only provide a reproducible method to obtain thymic epithelial cells (TECs) or TEC progenitors (TEPs) by inducing differentiation of human pluripotent stem cells into thymic epithelial cells (TECs) or TEC progenitors (TEPs) but also provide an increase the purity and homogeneity of the thymic epithelial cells (TECs), or TEC progenitors (TEPs) thus increasing function.

The methods and systems set forth herein generate a defined and reproducible cell population that is fully functional upon transplantation. Furthermore, the methods and systems set forth herein provide a substantially homogenous population of thymic epithelial cells (TECs) or TEC progenitors.

A human pluripotent stem cell is the starting material of the methods of the invention.

The human pluripotent stem cell (hPSCs) can be an embryonic stem cells (ESCs) or an induced pluripotent stem cell (iPSCs).

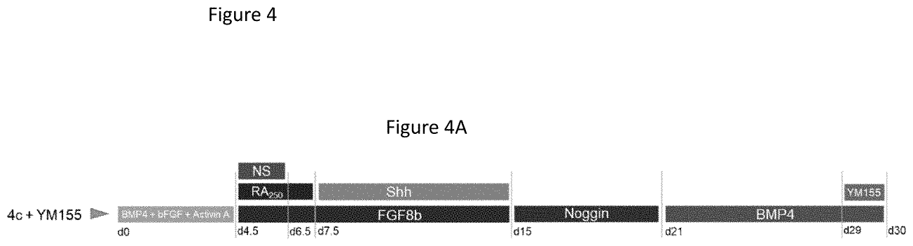

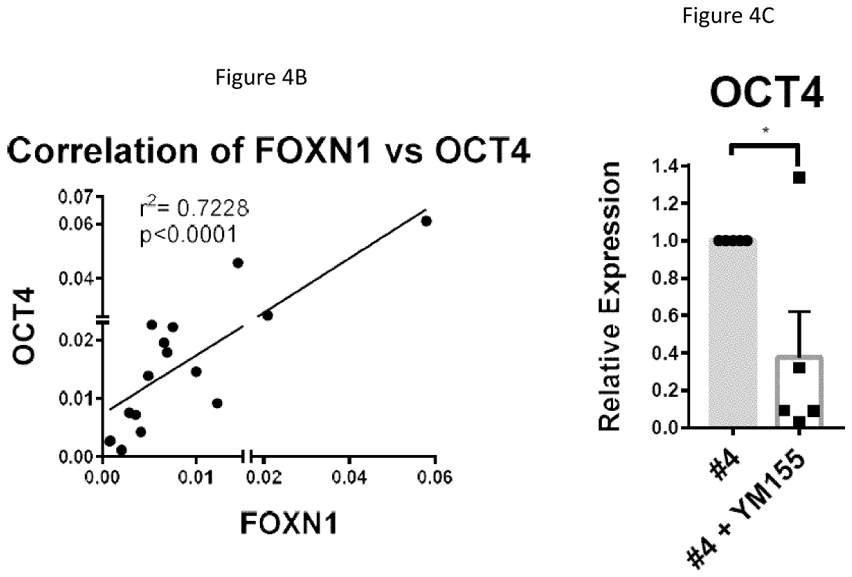

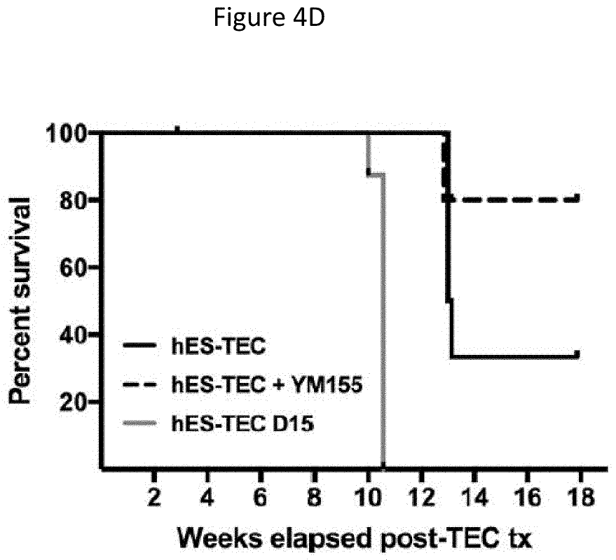

The steps of the method and the timing are set forth in Table 1 and FIG. 4A.

TABLE 1
Timeline of the Differentiation Method

| STEP | TIMING | GENERAL DESCRIPTION |
|---|---|---|
| 1 | Performed from about day 1 | Differentiate hPSCs to |
| | to about day 6 | definitive endoderm cells |
| 2 | Starting from about day 3 to | Differentiate definitive |
| | about day 5 and performed | endoderm cells to anterior |
| | for about 48 to about 72 | foregut endoderm cells by |
| | hours, ending at about day 5 | inhibiting BMP and/or TGFβ |
| | to day 8 | signaling and stimulating |
| | | expression of TBX1 and/or |
| | | optionally stimulating |
| | | HOXA3 |
| 3 | Starting from about day 5 to | Differentiate anterior foregut |
| | about day 8 and performed | endoderm cells to pharyngeal |
| | for about 6 days to about 10 | endoderm cells by continuing |
| | days ending at about day 11 | to stimulate expression of |
| | to about day 18 | HOXA3 (for about one day to |
| | | three days) and/or later |
| | | stimulating expression of |
| | | PAX1 and PAX9 and/or |
| | | stimulating TBX1 throughout |
| 4 | Starting from about day 11 to | Differentiate pharyngeal |
| | about day 18 and performed | endoderm cells into distal |
| | for about 4 days to about 7 | third PP, thymic epithelial |
| | days ending at about day 19 | cells, or thymic epithelial |
| | to about day 25 | progenitor cells by inhibiting |
| | | BMP |
| 5 | Starting from about day 13 to | Continue to differentiate |
| | about day 25 and performed | pharyngeal endoderm cells |
| | about 5 days to about 15 days | into distal third PP, thymic |
| | ending at about day 18 to | epithelial cells, or thymic |
| | about day 40 | epithelial progenitor cells by |
| | | adding BMP |
| 6 | Starting from about day 21 to | Add survivin inhibitor |
| | about day 23 or at the end of | |
| | the protocol for about 24 to | |
| | about 72 hours | |

The first step of the method is differentiating the hPSCs to definitive endoderm (DE) cells using any method known in the art. Exemplified here was the use of previously published protocols using serum-free differentiation medium containing BMP4, bFGF and Activin A. However, other protocols known in the art can be used.

The next step of the method is the culturing the resulting definitive endoderm cells from the first step to further differentiate into anterior foregut endoderm (AFE). Any medium used for differentiation protocols can be used for culturing the cells at this step. A serum-free differentiation medium is preferred. Additionally, growth factors such as EGF and FGF can be added to the medium to promote cellular growth.

The endoderm cells are then contacted or incubated with an agent that inhibits BMP and an agent that inhibits TGFβ signaling to promote differentiation of the definitive endoderm cells to anterior foregut progenitor cells. The most efficient method to accomplish this is by adding the agents to the medium in which the cells are being cultured. However, any other method known in the art that would contact or incubate the cells with the agents can be used. The cells can be contacted or incubated with the agents simultaneously or concurrently.

Agents that inhibit BMP include but are not limited to Noggin and Dorsomorphin. Agents that inhibit TGFβ signaling include but are not limited to SB431542.

Dorsomorphin can be used in an amount ranging from about 0.5 μM to about 2 M.

Noggin can be used in an amount ranging from about 25 ng/ml to about 500 ng/ml, or ranging from about 50 ng/ml to about 400 ng/ml, or ranging from about 100 ng/ml to about 300 ng/ml, with about 200 ng/ml being a preferred amount.

An agent for the inhibition of TGFβ signaling is SB431542 in an amount ranging from about 1 μM to about 50 μM, or ranging from about 2 μM to about 30 μM, or ranging from about M to about 20 μM. In some embodiments, the agent used for the inhibition of TGFβ signaling is SB431542 in the amount of about 10 μM.

However, other agents that inhibit TGFβ signaling can be used in the method.

Additionally, it was found that the combined stimulation of expression of TBX1 and HOXA3 at the AFE stage was essential for the physiological 3^{rd} PP endoderm development.

Thus, the cells are further contacted or incubated with agents which stimulate expression of these genes. An agent for the stimulation of TBX1 is FGF8b, which may be used in an amount ranging from about 10 ng/ml to about 200 ng/ml, or ranging from about 20 ng/ml to about 150 ng/ml, or ranging from about 30 ng/ml to about 100 ng/ml. In some embodiments, the FGF8b may be used at about 50 ng/ml.

The cells are contacted or incubated with this agent from about day 4.5 to about day 15.

An agent for the stimulation of HOXA3 is retinoic acid (RA) used in an amount ranging from about 0.1 μM to about 0.6 μM, or ranging from about 0.2 μM to about 0.5 μM. In some embodiments, the retinoic acid may be used in the amount of about 0.6 μM. The cells can be contacted or incubated with this agent from about day 4.5 to about day 7.5. Stimulation of HOXA3 can be performed at any other period of 3 days during the first 15 days, other than day 4.5 to 7.5.

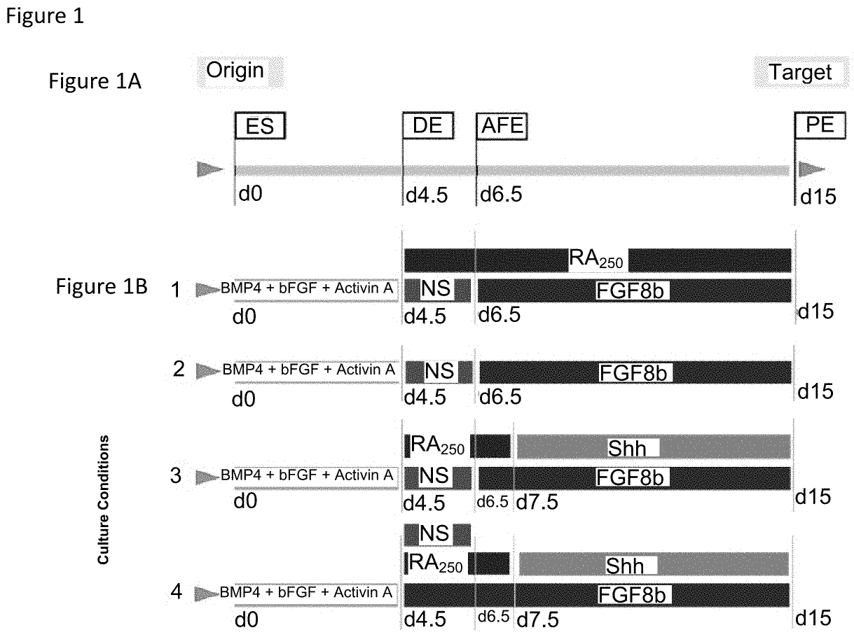

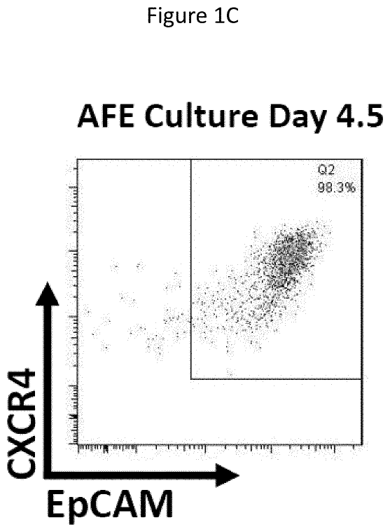

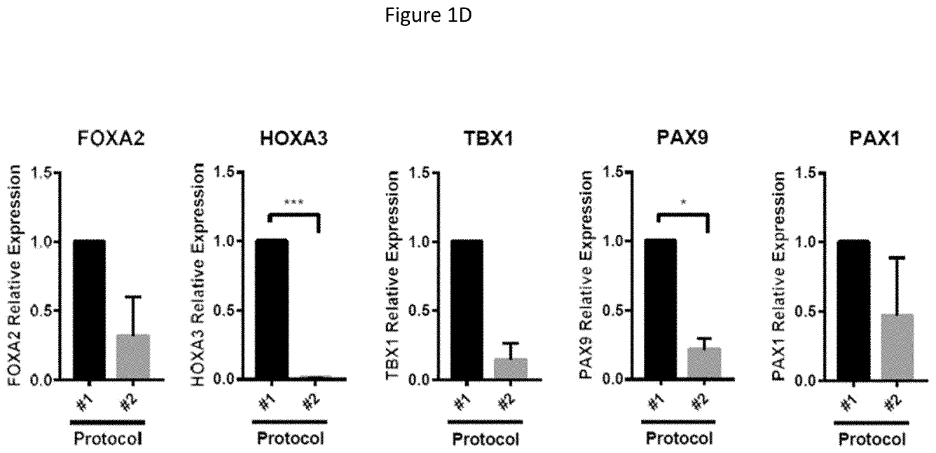

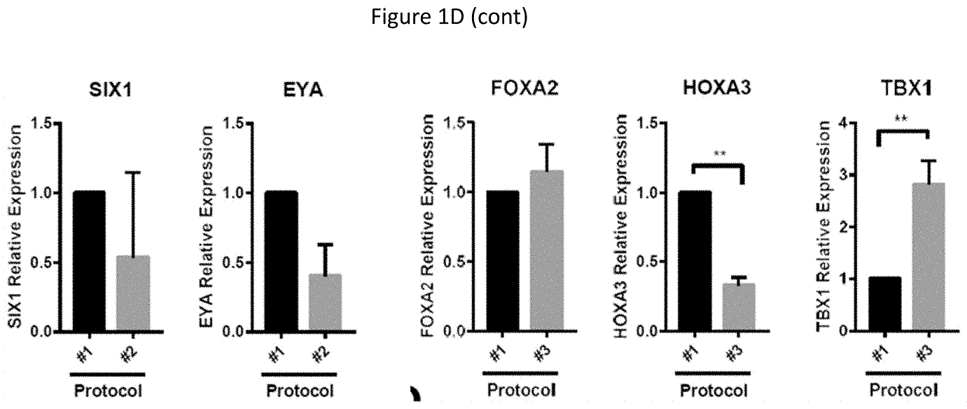

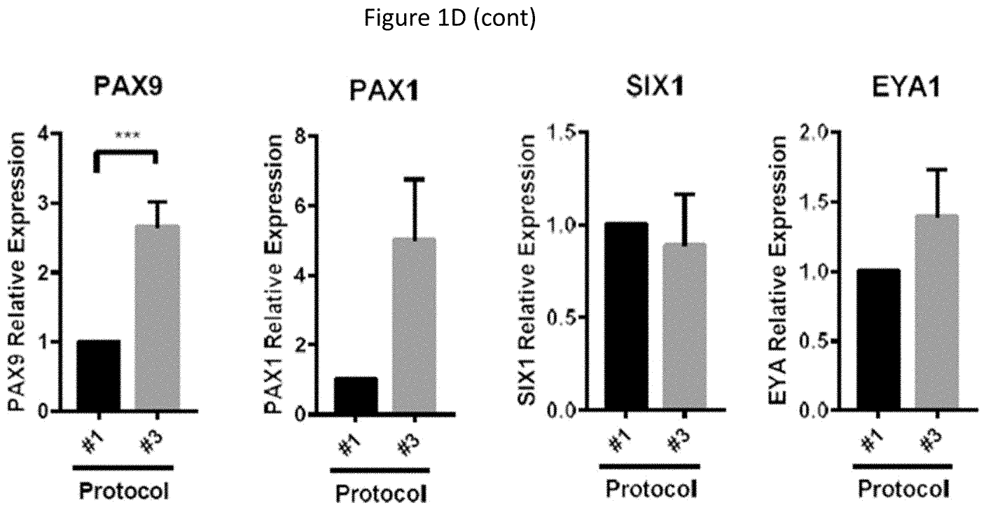

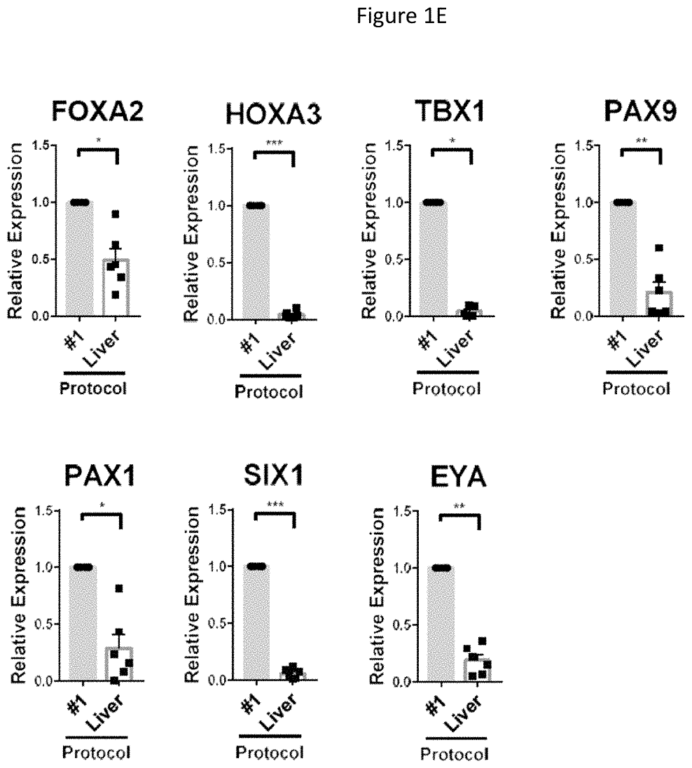

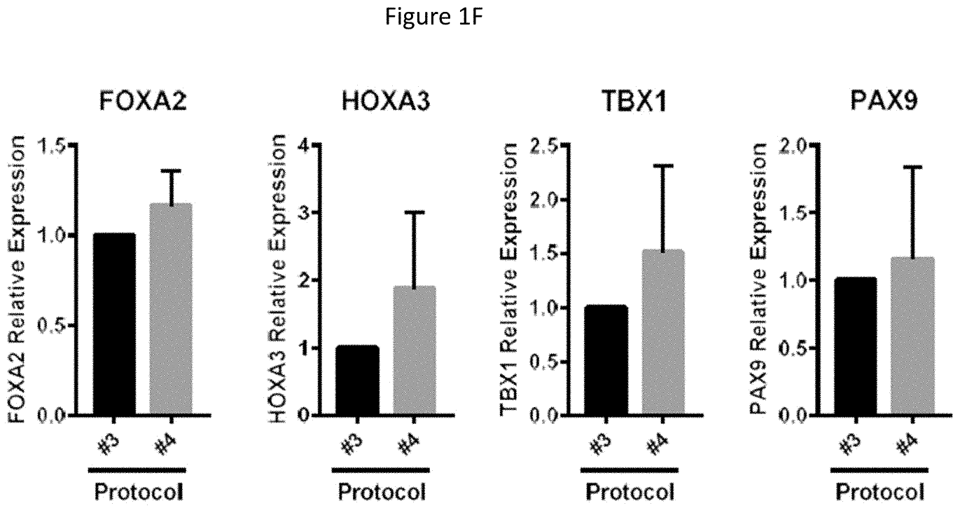

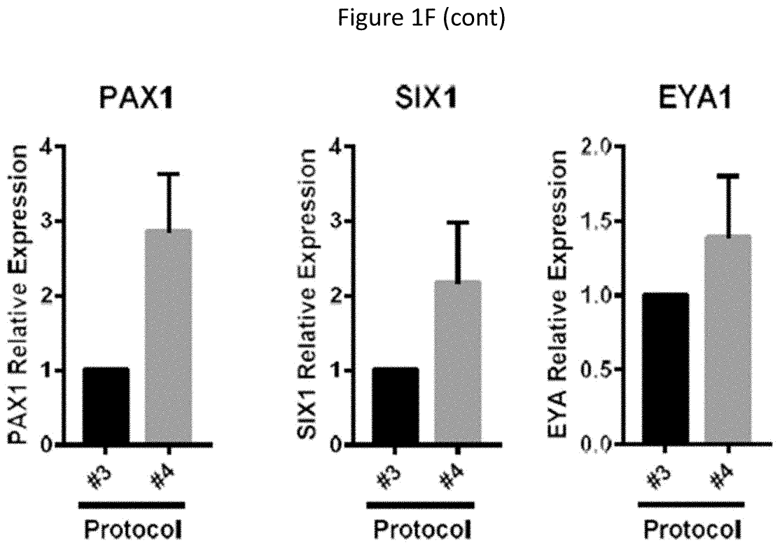

As shown in FIGS. 1D-1F, this protocol yields AFE with high efficiency.

The cells continue to be cultured in any serum-free medium used for differentiation of cells (herein referred to as the “differentiation medium” or “serum-free differentiation medium). Additionally, growth factors such as EGF and FGF can be added to the differentiation medium to promote cellular growth. At the beginning of this step for about one to two days, the cells are contacted or incubated with RA in an amount of ranging from about 0.1 μM to about 0.6 μM, or ranging from about 0.2 μM to about 0.5 μM. In some embodiments, the cells are contacted or incubated with about 0.25 μM RA. Also the cells continue to be contacted or incubated with FGF8b throughout this step, in an amount ranging from about 10 ng/ml to about 200 ng/ml, or ranging from about 20 ng/ml to about 150 ng/ml, or ranging from about 30 ng/ml to about 100 ng/ml. As a non-limiting example, the cells may be contacted with about 50 ng/ml FGF8b.

The next step promotes differentiation of the anterior foregut cells into pharyngeal endoderm (PE) cells.

In this step, the cells are contacted or incubated with an agent that induces expression of PAX9 and PAX1. The most efficient method to accomplish this is by adding the agents to the medium in which the cells are being cultured. However, any other method known in the art that would contact or incubate the cells with the agents can be used. The cells can be contacted or incubated with the agents simultaneously or concurrently. An agent for the stimulation of both PAX9 and PAX1 is sonic hedgehog (Shh) in an amount ranging from about 10 ng/ml to about 400 ng/ml, or ranging from about 25 ng/ml to about 300 ng/ml, or ranging from about 50 ng/ml to about 200 ng/ml. In some embodiments, Shh may be used at about 100 ng/ml.

Also the cells are continued to be contacted or incubated with FGF8b throughout at an amount ranging from about 10 ng/ml to about 200 ng/ml, or ranging from about 20 ng/ml to about 150 ng/ml, or ranging from about 30 ng/ml to about 100 ng/ml. In some embodiments, cells may be contacted or incubated with about 50 ng/ml FGF8b.

Noggin can also be used to induce expression of PAX9 and PAX1. Noggin can be used in an amount ranging from about 50 ng/ml to about 400 ng/ml, or ranging from about 60 ng/ml to about 300 ng/ml, or ranging from about 75 ng/ml to about 200 ng/ml. In some embodiments, Noggin may be used in the amount of about 100 ng/ml.

This step is performed for about 4 to about 10 days.

The next step is the differentiation of the PE cells to distal third PP/TECs. This step is divided into two steps: the first where the cells are contacted or incubated with an agent which inhibits BMP. Agents which inhibit BMP include but are not limited to Noggin and Dorsomorphin.

Dorsomorphin can be used in an amount ranging from about 0.5 μM to about 2 M.

Noggin can be used in an amount ranging from about 50 ng/ml to about 400 ng/ml, or ranging from about 60 ng/ml to about 300 ng/ml, or ranging from about 75 ng/ml to about 200 ng/ml. As a non-limiting example, Noggin may be used in the amount of about 100 ng/ml.

This part of the step is performed for about 5 days to about 7 days.

The second part of the step the cells are contacted or incubated with BMP4 in an amount ranging from about 5 ng/ml to about 300 ng/ml, or ranging from about 15 ng/ml to about 200 ng/ml, or ranging from about 25 ng/ml to about 100 ng/ml, or with about 50 ng/ml. This part of the step is performed for about 5 days to about 10 days.

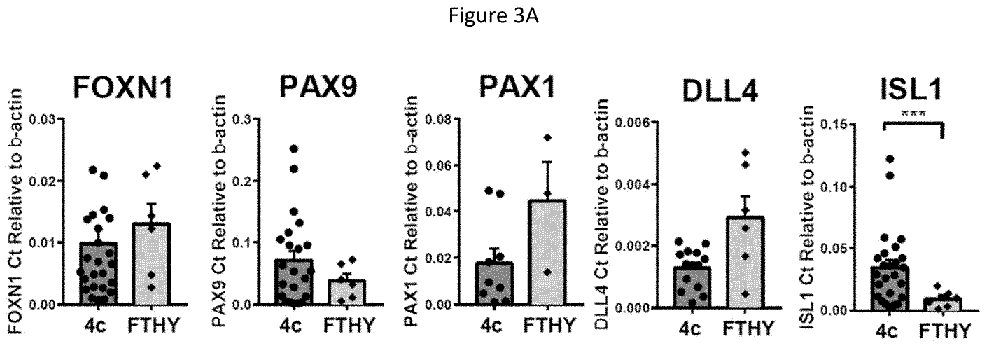

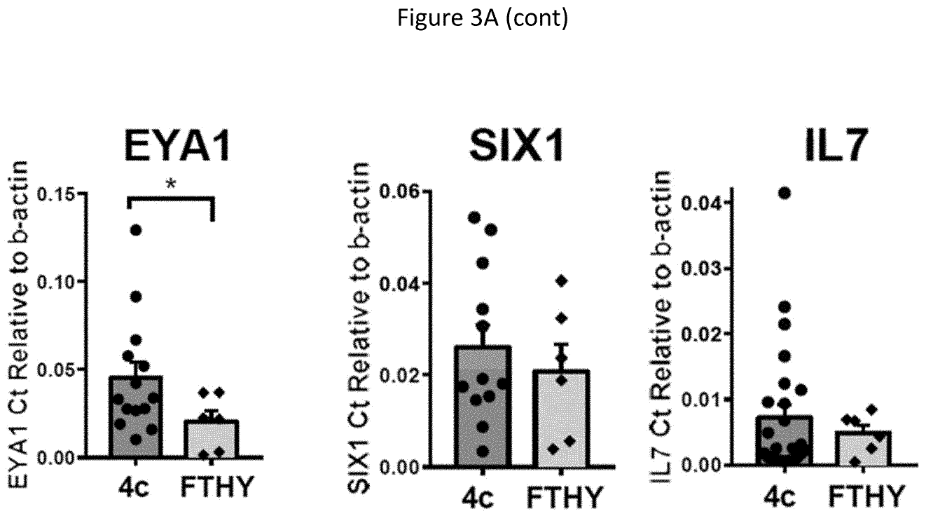

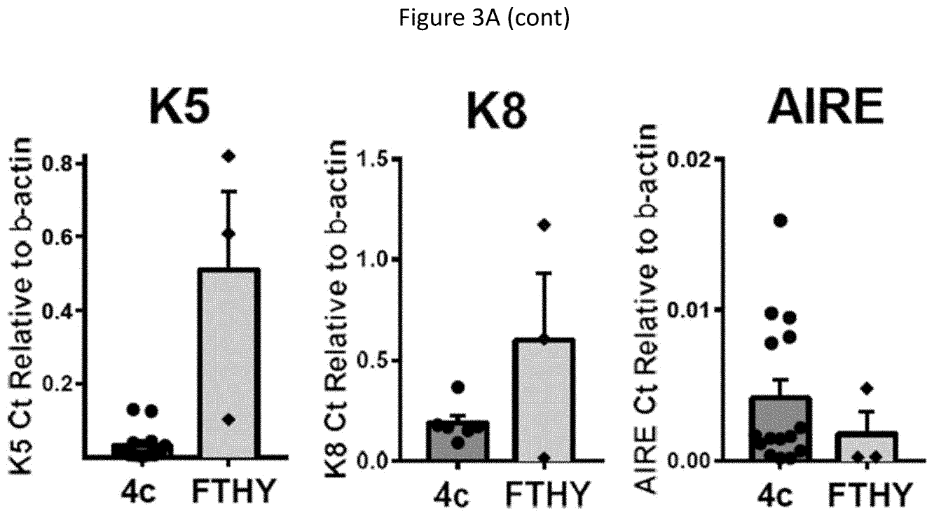

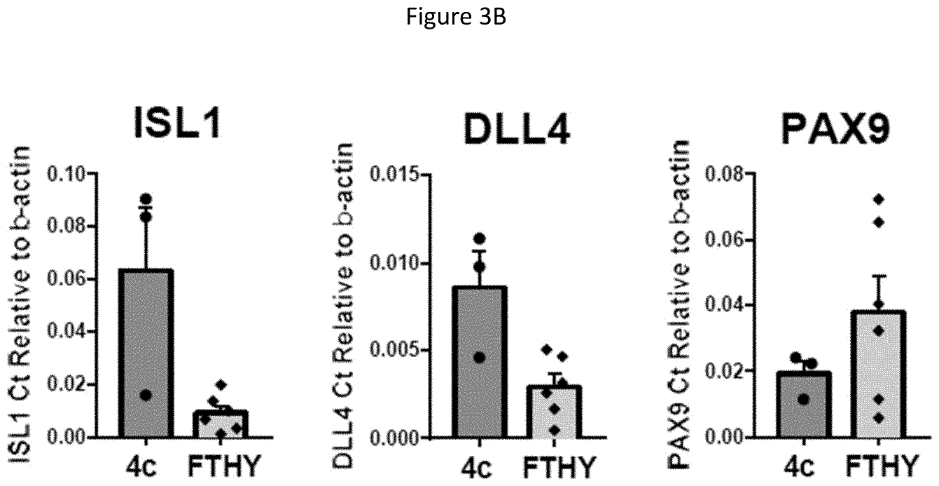

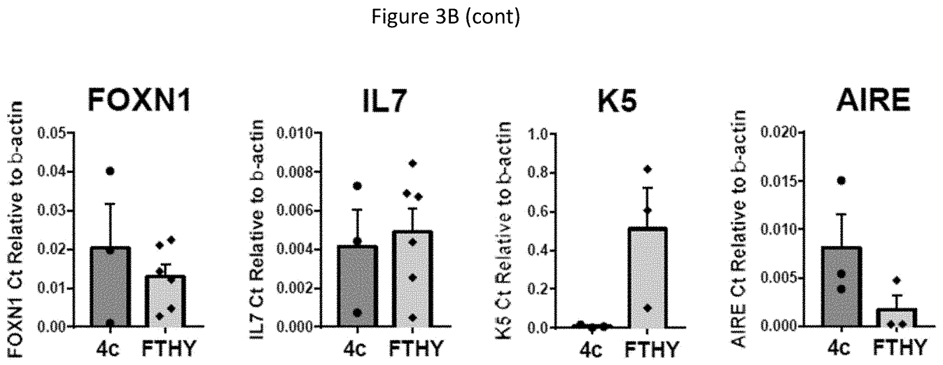

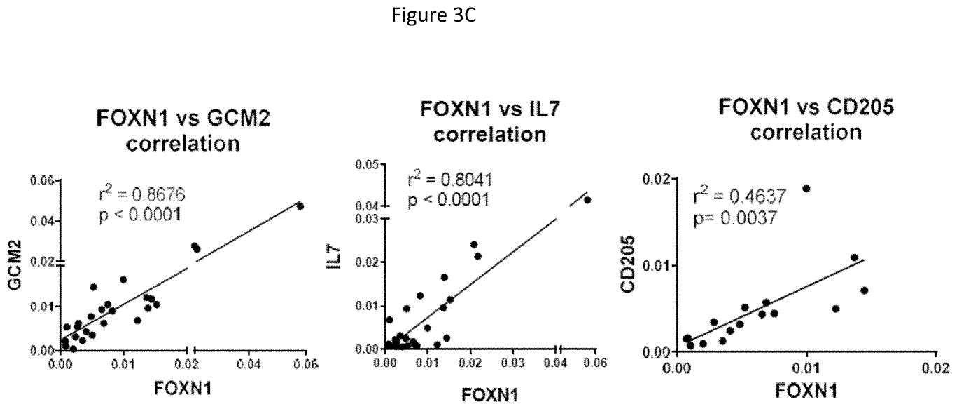

The final cells obtained following the method may show gene expression of TEC markers including FOXN1, PAX9, PAX1, DLL4, ISL1, EYA1, SIX1, IL7, K5, K8 and AIRE. See FIGS. 3A and 3B.

While the method set forth above is a novel, reproducible and robust method to induce the differentiation of hPSCs to TECs or TEPs, the present method also provides for further steps to reduce and eliminate pluripotent cells which can cause teratomas in the final grafted cells. In this step the cells are contacted or incubated with a survivin inhibitor such as YM155 for about the last 24 hours of the method in an amount ranging from about 5 nM to about 50 nM. As a non-limiting example, cells may be contacted or incubated with 20 nM of YM155. The cells may also be contacted or incubated with a survivin inhibitor concurrent with the BMP4 treatment. In some embodiments, the cells may be contacted or incubated with a survivin inhibitor during the first 24 to 48 hours of concurrent BMP4 incubation.

The present invention also includes systems for practicing the disclosed methods for obtaining TECs or TEPs from hPSCs. These systems can include subsystems wherein the subsystems include differentiation medium, and agents which inhibit BMP and TGFβ signaling, agents which stimulate expression of HOXA3, TBX1, PAX1 and PAX9, agents which inhibit surviving, and BMP4. These systems can include subsystems wherein the subsystems include differentiation medium, and Noggin, retinoic acid, FGF8b, sonic hedgehog, BMP, and YM155.

### Cells

A further embodiment of the present disclosure are the thymic epithelial cells (TECs) or TEC progenitors (TEPs) generated by the differentiation protocol set forth herein.

In some embodiments, these cells express FOXN1, EpCAM, Keratin 5, and Keratin 8. In some embodiments, these cells are thymic epithelial cells (TECs). In some embodiments, these cells are thymic epithelial cell progenitors (TEC progenitors) (TEPs).

Thus, one aspect of the present disclosure is thymic epithelial cells (TECs) or TEC progenitors (TEPs) suitable for administration, transplantation and grafting into a subject produced by the methods as described herein.

In another aspect, provided herein is a composition comprising the thymic epithelial cells or TEC progenitors (TEPs) produced by the methods as described herein. In some embodiments, these cells are suitable for administration, transplantation and grafting into a subject.

In some embodiments, the composition is a pharmaceutical composition further comprising any pharmaceutically acceptable carrier or excipient.

In certain embodiments, the composition or pharmaceutical composition comprises at least 10,000, at least 50,000, at least 100,000, at least 500,000, at least 1×10⁶, at least 5×10⁶, at least 1×10⁷, at least 5×10⁷, at least 1×10⁸, at least 5×10⁸, at least 1×10⁹, at least 5×10⁹, or at least 1×10¹⁰ thymic epithelial cells (TECs) or TEC progenitors (TEPs) produced by the methods as described herein. In some embodiments, these cells are suitable for administration, transplantation and grafting into a subject.

In certain embodiments, the disclosure provides a cryopreserved composition or solution of the thymic epithelial cells (TECs) or TEC progenitors (TEPs) produced by the methods as described herein. In some embodiments, these cells are suitable for administration, transplantation and grafting into a subject.

In certain embodiments, the cryopreserved composition or solution comprises at least 10,000, at least 50,000, at least 100,000, at least 500,000, at least 1×10⁶, at least 5×10⁶, at least 1×10⁷, at least 5×10⁷, at least 1×10⁸, at least 5×10⁸, at least 1×10⁹, at least 5×10⁹, or at least 1×10¹⁰ thymic epithelial cells (TECs) or TEC progenitors (TEPs) produced by the methods as described herein. In some embodiments, these cells are suitable for administration, transplantation and grafting into a subject.

In certain embodiments, the disclosure provides for cell culture comprising thymic epithelial cells (TECs) or TEC progenitors (TEPs) produced by the methods as described herein. In certain embodiments, the cell culture comprises at least 1×10⁷, at least 5×10⁷, at least 1×10⁸, at least 5×10⁸, at least 1×10⁹, at least 5×10⁹, or at least 1×10¹⁰ thymic epithelial cells (TECs) or TEC progenitors (TEPs) produced by the methods as described herein. In some embodiments, these cells are suitable for administration, transplantation and grafting into a subject.

In certain embodiments, the disclosure provides the therapeutic use of the thymic epithelial cells (TECs) or TEC progenitors (TEPs) suitable for administration, transplantation and grafting into a subject produced by the methods as described herein, and compositions, solutions and cell cultures comprising such cells.

In other embodiments, the disclosure provides for a population of substantially homogenous thymic epithelial cells (TECs) or TEC progenitors (TEPs) produced by the methods as described herein. In some embodiments, these cells are suitable for administration, transplantation and grafting into a subject. In some embodiments, the population of cells comprises at least about 90%, 91%, 92%, 93%, 94%, 95%, 96%, 97%, 98%, or 99% thymic epithelial cells (TECs) or TEC progenitors (TEPs).

In another aspect, provided herein is a composition comprising the population of substantially homogenous thymic epithelial cells (TECs) or TEC progenitors (TEPs) produced by the methods as described herein. In some embodiments, these cells are suitable for administration, transplantation and grafting into a subject. In some embodiments, the composition is a pharmaceutical composition further comprising any pharmaceutically acceptable carrier or excipient.

In certain embodiments, the population or composition or pharmaceutical composition comprises at least 10,000, at least 50,000, at least 100,000, at least 500,000, at least 1×10⁶, at least 5×10⁶, at least 1×10⁷, at least 5×10⁷, at least 1×10⁸, at least 5×10⁸, at least 1×10⁹, at least 5×10⁹, or at least 1×10¹⁰ thymic epithelial cells (TECs) or TEC progenitors (TEPs) produced by the methods as described herein. In some embodiments, these cells are suitable for administration, transplantation and grafting into a subject.

In certain embodiments, the disclosure provides a cryopreserved composition or solution of the population of substantially homogenous thymic epithelial cells (TECs) or TEC progenitors (TEPs) produced by the methods as described herein. In certain embodiments, the cryopreserved composition or solution comprises at least 10,000, at least 50,000, at least 100,000, at least 500,000, at least 1×10⁶, at least 5×10⁶, at least 1×10⁷, at least 5×10⁷, at least 1×10⁸, at least 5×10⁸, at least 1×10⁹, at least 5×10⁹, or at least 1×10¹⁰ thymic epithelial cells (TECs) or TEC progenitors (TEPs) produced by the methods as described herein. In some embodiments, these cells are suitable for administration, transplantation and grafting into a subject.

In certain embodiments, the disclosure provides for cell culture comprising population of substantially homogenous thymic epithelial cells (TECs) or TEC progenitors (TEPs) produced by the invention as described herein. In certain embodiments, the cell culture comprises at least 1×10⁷, at least 5×10⁷, at least 1×10⁸, at least 5×10⁸, at least 1×10⁹, at least 5×10⁹, or at least 1×10¹⁰ thymic epithelial cells (TECs) or TEC progenitors (TEPs) produced by the methods as described herein. In some embodiments, these cells are suitable for administration, transplantation and grafting into a subject.

In certain embodiments, the disclosure provides the therapeutic use of the population of substantially homogenous thymic epithelial cells (TECs) or TEC progenitors (TEPs) suitable for transplantation and grafting into a subject produced by the methods as described herein, and compositions, solutions and cell cultures comprising such cells.

A further embodiment is a thymic organ comprising the TECs or TEPs disclosed herein combined with other cells which make up a thymus.

### Therapeutic Uses

The novel method described herein for the generation of TECs or TEC progenitors (TEPs) from stem cells and the cells and substantially homogenous population of cells generated from this method, provide new therapies for diseases.

The ability to generate functional TECs from human pluripotent stem cells, would have important applications in modeling human immune responses in mice, and in modeling and treating thymus deficiency syndromes, such as DiGeorge syndrome, Nude syndrome, and immunodeficiency complicating bone marrow transplantation for leukemia. Cells could also be used clinically for cell-therapy and transplanted in patients to achieve T cell reconstitution, or generating immune tolerance to prevent graft rejection after an organ transplantation, or for recovering an impaired thymic functionality due to injuries or aging

Thus, one embodiment is a method of treating or preventing a disease of the thymus in a subject in need thereof comprising the steps of administering, transplanting or grafting a therapeutically effective amount of the cells of the present disclosure, a solution comprising the cells of the present disclosure, a composition comprising the cells of the present disclosure, or a pharmaceutical composition comprising the cells of the present disclosure, to the subject in need thereof. The subject is preferably a mammal, and most preferably human.

A further embodiment is a method of treating or preventing an autoimmune disease in a subject in need thereof comprising the steps of administering, transplanting or grafting a therapeutically effective amount of the cells of the present disclosure, a solution comprising the cells of the present disclosure, a composition comprising the cells of the present disclosure, or a pharmaceutical composition comprising the cells of the present disclosure, to the subject in need thereof. The subject is preferably a mammal, and most preferably human.

Another embodiment is a method of recovering or restoring impairment of the function of the thymus in a subject in need thereof comprising the steps of administering, transplanting or grafting a therapeutically effective amount of the cells of the present disclosure, a solution comprising the cells of the present disclosure, a composition comprising the cells of the present disclosure, or a pharmaceutical composition comprising the cells of the present disclosure, to the subject in need thereof. The subject is preferably a mammal, and most preferably human. In some embodiments the impairment is due to injury. In some embodiments, the impairment is due to aging. In some embodiments, the impairment is due to congenital abnormalities.

Yet a further embodiment is a method of reconstituting T cells after a bone marrow transplant in a subject in need thereof comprising the steps of administering, transplanting or grafting a therapeutically effective amount of the cells of the present disclosure, a solution comprising the cells of the present disclosure, a composition comprising the cells of the present disclosure, or a pharmaceutical composition comprising the cells of the present disclosure, to the subject in need thereof. The subject is preferably a mammal, and most preferably human.

The cells obtained using the methods disclosed herein can be used to generate a hybrid thymus. In some embodiments the hybrid thymus comprises thymic epithelial cells obtained using the methods disclosed herein and thymic tissue from a second individual of the same species. In some embodiments the hybrid thymus comprises thymic epithelial cells obtained using the methods disclosed herein and thymic tissue from a second species. In some embodiments, the second species is a swine. In some embodiments, the second species is a miniature swine. In some embodiments, the swine a juvenile swine. In some embodiments, the swine is fetal. A method of obtaining such a hybrid swine is disclosed in commonly owned patent application no. PCT/US2019/051865.

A further embodiment is the use of the cells to develop mice models. Since cellular reprogramming was discovered (iPSCs), a new era of disease modelling with pluripotent stem cells representing a myriad of genetic diseases can now be produced from patient tissue. IPSCs from patients with different autoimmune diseases where the central tolerance is involved can be differentiated to TECs (or TEPs), then injected or grafted into mice where the cells can reproduce and develop into the various conditions or disorders. Humanized mouse models can be generated from TECs from patients with an autoimmune disease such as multiple sclerosis, or type I diabetes, or a congenic abnormality such as DiGeorge Syndrome. The mouse, in vivo environment can then be used to study the progress of a disorder that, otherwise, could not be developed in vitro.

Additionally, personalized humanized mouse models can be generated using the cells described herein. Thus far, the most developed humanized mouse model contains human hematopoietic stem cells (HSCs), and a sample of a pediatric or human fetal thymus sample grafted under the kidney capsule. The limitation of these mouse models is that the HLA from both type of cell populations (HSCs and TECs) do not match because they originate from two different individuals. With the differentiation protocol disclosed herein, TECs (or TEPs) could be differentiated from the same iPSCs as the HSCs, so the immune system cells HLA will match with the ones on the human TECs transplanted on the mouse. This technology could be used for individual patients resulting in a Personalized Immune (PI) mouse.

A further embodiment is the use of the cells for drug testing in vivo (with the previously described mouse models including but not limited to the Personalized Immune (PI) mouse model) or in vitro. In vitro, differentiated TECs cultures, can be used to test drugs against different conditions that affect to TECs, such as cancer (thymomas), or infectious, or autoimmune diseases.

### Kits

The present disclosure also provides kits.

In one embodiment, the kit includes one or more components including human pluripotent stem cells, medium for culturing and differentiation the hPSCs, such medium including growth factors and inhibit BMP and TGFβ signaling, agents which stimulate expression of HOXA3, TBX1, PAX1 and PAX9, agents which inhibit surviving, and BMP4.

In another embodiment, the kit includes one or more components including human pluripotent stem cells, medium for culturing and differentiation the hPSCs, such medium including growth factors and Noggin, retinoic acid, FGF8b, sonic hedgehog, BMP, and YM155.

In further embodiments, a kit can include the TECs or TEC progenitors (TEPs) obtained by the current methods and systems of the disclosure. The kit can also comprise reagents for culturing the cells.

In further embodiments, a kit can include a pharmaceutical composition comprising the TECs or TEC progenitors (TEPs) obtained by the current methods and systems of the disclosure.

In further embodiments, a kit can include a cryopreserved composition comprising the TECs or TEC progenitors (TEPs) obtained by the current methods and systems of the disclosure.

The kits can further include a package insert including information concerning the pharmaceutical compositions and dosage forms in the kit. For example, the following information regarding a combination of the invention may be supplied in the insert: how supplied, proper storage conditions, references, manufacturer/distributor information and patent information.

### EXAMPLES

The present invention may be better understood by reference to the following non-limiting examples, which are presented in order to more fully illustrate the preferred embodiments of the invention. They should in no way be construed to limit the broad scope of the invention.

### Example 1—Materials and Methods

Maintenance of hPSCs

RUES2 (Rockefeller University Embryonic Stem Cell Line 2, NIH approval number NIHhESC-09-0013, Registration number 0013; passage 13-24) were cultured on mouse embryonic fibroblasts as previously described (Green et al. 2011). Mouse embryonic fibroblasts (GlobalStem, Rockville, Md.) were plated at a density of approximately 25,000 cells/cm². hPSCs were cultured in DMEM/F12 with 20% knockout serum replacement [Gibco (Life Technologies, Grand Island, N.Y.)], 0.1 mM β-mercaptoethanol (Sigma-Aldrich, St. Louis, Mo.), and 20 ng/ml FGF-2 (R&D Systems, Minneapolis, Minn.). Medium was changed daily and cells were passaged with accutase/EDTA (Innovative Cell Technologies, San Diego, Calif.) every 4 days at 1:24 dilution. Undifferentiated hPSCs were maintained in a 5% C02/air environment. Human H9 ES cell line was also treated with protocol #4c. Lines were karyotyped and verified for mycoplasma contamination using PCR every 6 months.

### Induction of Endoderm

The differentiation was performed as described Huang et al. 2014 in serum-free differentiation (SFD) medium consisting of DMEM/F12 (3:1) (Life Technologies) supplemented with N2 [Gibco (Life Technologies)], B27 (Gibco), ascorbic acid (50 μg/ml, Sigma), Glutamax (2 mM, Life Technologies), monothioglycerol (0.4 μM, Sigma), 0.05% bovine serum albumin (BSA) (Life Technologies) and 1% penicillin-streptomycin (Thermo Fisher Scientific, Waltham, Mass.). Cells were then briefly trypsinized (0.05%, 1 min at 37° C.) into single cell suspension and plated onto low attachment 6-well plates [Costar 2 (Corning Incorporated, Tewksbury Mass.)] to form embryoid bodies in serum-free differentiation medium containing human BMP4, 0.5 ng/ml, human bFGF, 2.5 ng/ml (R&D Systems) and human activin A, 100 ng/ml (R&D Systems) for 84 hours (3.5 days approximately) on low-adherence plates. Embryoid bodies were then collected, briefly trypsinized (0.05%, 1 min at 37° C.) into 3-10 small cell clumps and resuspended again in endoderm induction medium for another 24 hours. Cells were fed every 24-48 hr (depending on the density) and maintained in a 5% CO₂/5% O₂/90% N₂ environment.

### Induction of Anterior Foregut Endoderm, Pharyngeal Endoderm and Distal 3rd Pharyngeal Pouch

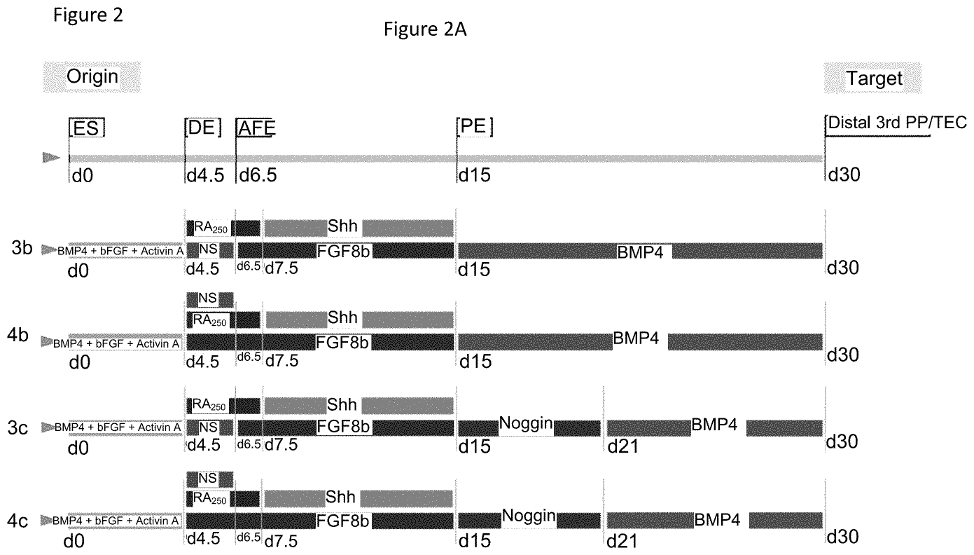

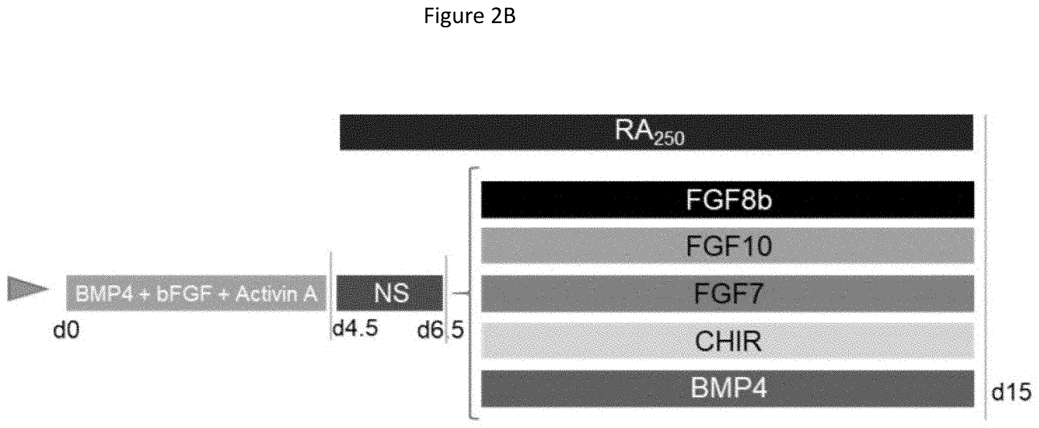

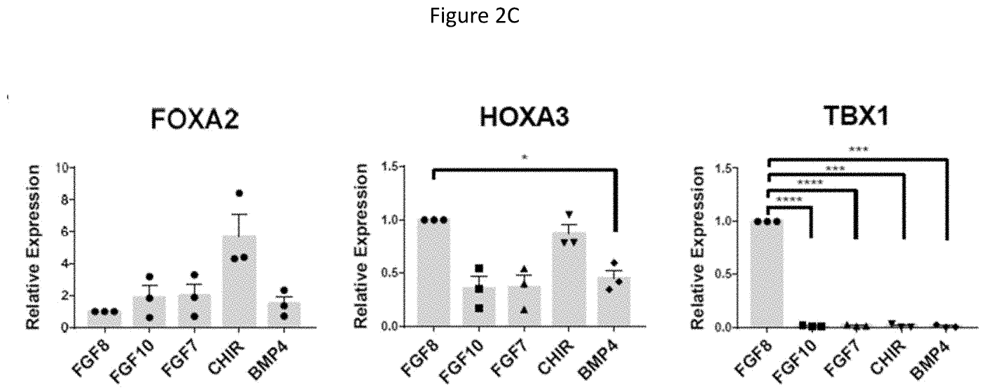

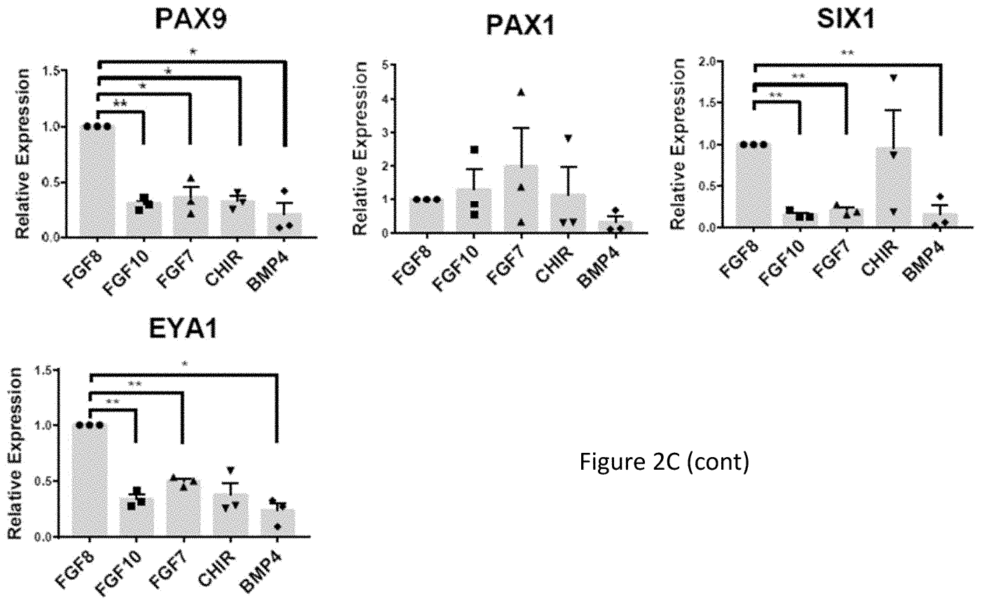

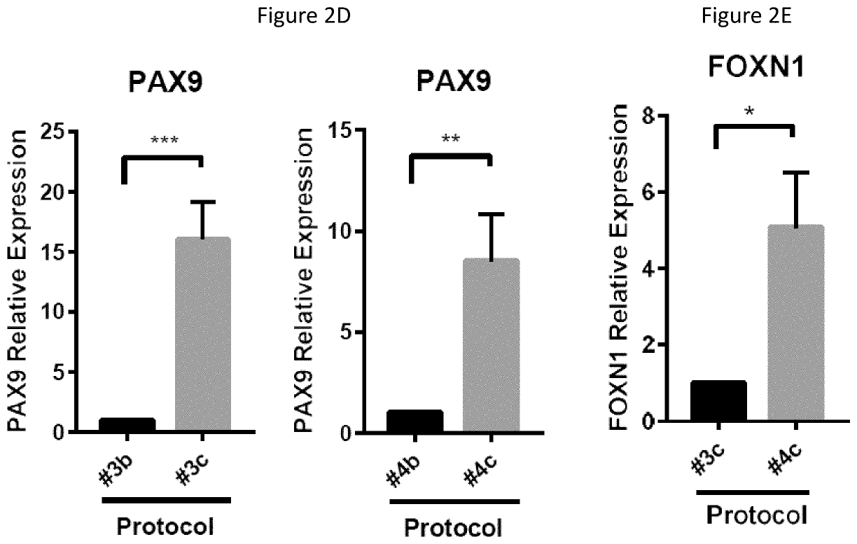

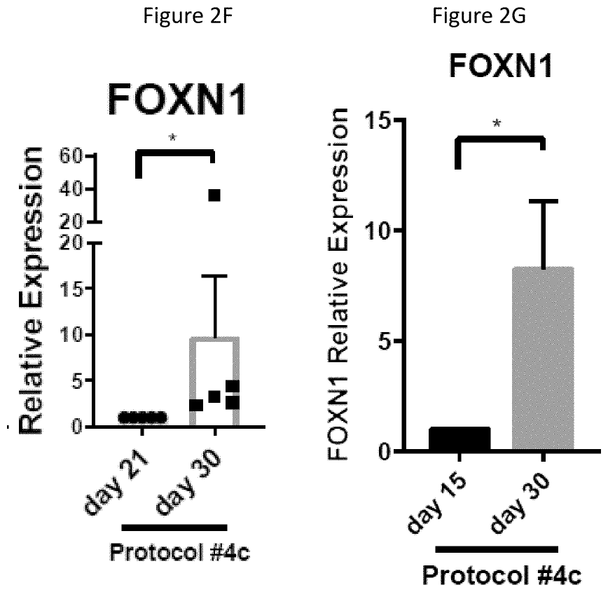

After 108 hours in total on low-adherence plates with endoderm induction media (described above), embryoid bodies were collected and, without being trypsinized, plated on matrigel-coated, 24-well tissue culture plates (approximately 50,000-70,000 cells/well) in SFD medium supplemented with 200 ng/mL recombinant human (rh) Noggin and 10 μM SB431542 (NS) (as described in established protocols Green et al. 2011), with Retinoic Acid (0.25 μM) and FGF8b 50 ng/mL (as a novel modification of this protocol) for 48 hours. For pharyngeal endoderm, the resulting cells were then treated for 24 hours with FGF8b (50 ng/mL) and Retinoic Acid (0.25 M) followed by 8 days with FGF8b (50 ng/mL) and Sonic Hedgehog (Shh) (100 ng/mL) (FIG. 1B). For the 3rd pharyngeal pouch specification, cells were then exposed to rhNoggin (200 ng/mL) for 6 days, and then to BMP4 (10 ng/mL) until day 30 of differentiation (FIG. 2A). To avoid the formation of teratomas after engrafting in mice, cells were also exposed to survivin inhibitor YM155 (20 nM) (Lee et al. 2013)) for 24 hours during the last step in the later experiments (FIG. 4A). During the entire process, cell cultures were maintained in a 5% C02/air environment at 37° C. Cells were fed every 24 hours.

Quantitative Real-Time PCR Total RNA from clusters of ES cells differentiated for the indicated time with the indicated culture method was extracted using Trizol (Invitrogen), and Direct-zol RNA Miniprep Kit (Zymo Research) according to the manufacturer's instructions. NanoDrop 2000 spectrophotometer (ThermoFisher Scientific) was used to determine RNA concentration. 500 ng RNA was amplified with random hexamers by reverse transcription using Superscript III kit (Invitrogen) according to the manufacturer's instructions. Real-time quantitative PCR was performed in 20 ul volume using ABI Power SYBR Green PCR Master Mix on an ABI ViiA7 Thermocycler (Applied Biosystems Life Technologies). PCR cycling conditions were set at 50° C. for 2 minutes, 95° C. for 10 minutes followed by 95° C. for 15 seconds, and 60° C. for 1 minute for 40 cycles. Single peak dissociation/melting curve was verified for all reactions and primer pairs. Quantification of each gene transcript was obtained by comparing the average of triplicate experimental CT values to a standard curve of serially diluted genomic DNA for each primer target and then normalized by dividing by the CT housekeeping gene b-Actin. Primer sequences are listed in Table 2.

TABLE 2
Quantitative PCR Primers

| | Forward Primer | Reverse Primer |
|---|---|---|
| Gene | (5′-3′) | (5′-3′) |
| ACTB | TTTGAATGATGAGCCTTCGT | GGTCTCAAGTCAGTGTACAGGTA |
| | GCCC (SEQ ID NO: 1) | AGC (SEQ ID NO: 2) |
| K8 | CACCACAGATGTGTCCGAGA | AGGGCTGACCGACGAGAT |
| | (SEQ ID NO: 3) | (SEQ ID NO: 4) |
| EYA1 | ACCTCTGCCTTTGTGGTGAAT | GGCAGACACATAACGCTGTGCTA |
| | GGA (SEQ ID NO: 5) | AA (SEQ ID NO: 6) |
| FOXA2 | TTCAACCACCCGTTCTCCATC | CTGTTCGTAGGCCTTGAGGTCC |
| | AAC (SEQ ID NO: 7) | ATTT (SEQ ID NO: 8) |
| FOXN1 | CGGCACAACCTATCCCTCAA | TTGTCGATCTTGGCCGGATT |
| | (SEQ ID NO: 9) | (SEQ ID NO: 10) |
| HOXA3 | AGAGTTCCACTTCAACCGCT | ATGCCCTTGCCCTTCTGATCCT |
| | ACCT (SEQ ID NO: 11) | TT (SEQ ID NO: 12) |
| OCT3/4 | ATGCACAACGAGAGGATTTT | CTTTGTGTTCCCAATTCCTTCC |
| | GA (SEQ ID NO: 13) | (SEQ ID NO: 14) |
| PAX1 | AAACCCTCCATGAACTGTCC | CCCTGTGCTCCCTACTCCTACC |
| | TCTCC (SEQ ID NO: 15) | (SEQ ID NO: 16) |
| PAX9 | GGAAGCCGTGACAGAATGAC | TGGTTATGTTGCTGGACATGGG |
| | TACCT (SEQ ID NO: 17) | TG (SEQ ID NO: 18) |
| SIX1 | CTATTCTCTCCCGGGCTTAAC | CAGAGAGTCTTGGAGCTGATG |
| | (SEQ ID NO: 19) | (SEQ ID NO: 20) |
| TBX1 | CCCGGCTCCTACGACTATTG | GGAACGTATTCCTTGCTTGCCC |
| | C (SEQ ID NO: 21) | TT (SEQ ID NO: 22) |
| AIRE | CCTGGATGCACTTCTTGGA | CAGAGAGCTGTGGCCATGT |
| | (SEQ ID NO: 23) | (SEQ ID NO: 24) |
| CD205 | ATTGCTGGCACAGTACAGGA | TGGAATTCATGGACCTCCACTT |
| | (SEQ ID NO: 25) | (SEQ ID NO: 26) |
| DLL4 | GGGCACCTACTGTGAACTCC | GCTGCCCACAAAGCCATAAG |
| | (SEQ ID NO: 27) | (SEQ ID NO: 28) |
| K5 | TACCAGGACTCGGCTTCTGT | ATCATCGCTGAGGTCAAGGC |
| | (SEQ ID NO: 29) | (SEQ ID NO: 30) |
| IL7 | TCCTCCACTGATCCTTGTTC | CTTCAACTTGCGAGCAGCAC |
| | (SEQ ID NO: 31) | (SEQ ID NO: 32) |
| ISL1 | TTGTACGGGATCAAATGCGC | CCACACAGCGGAAACACTCGAT |
| | CAAG (SEQ ID NO: 33) | (SEQ ID NO: 34) |

### Immunohistochemistry and Immunofluorescence

hES-cultures in 24-well tissue culture plates were fixed with paraformaldehyde in PBS (4%) for 10 minutes at room temperature. Cells were washed in PBS twice, permeabilized in PBS with 0.1% triton for 20 min, and blocked in 5% fetal donkey serum for 1 hour at room temperature.

Thymic grafts were extracted, embedded in OCT (Tissue-Tec, Torrance Calif.) media, frozen and 5-7 um thick sections cut for immune staining. Sections were stained with H&E to visualize gross histology and interface of the thymic graft with the mouse renal tissue. For immunofluorescent staining, tissue sections were fixed and permeabilized in 100% ice-cold acetone and allowed to dry completely. Tissue sections were blocked in PBS supplemented with 0.1% Tween and 0.1% Bovine Serum Albumin. Slides were washed in PBS 0.1% Tween and stained with primary antibody for 2 hours at room temperature, and then washed and incubated in secondary antibodies for 2 hours at room temperature.

Cultures or tissue sections were incubated with one, or a combination of two or three of the following primary antibodies and appropriate secondary antibodies listed in Table 3. Images were collected on a Leica SCN 400 whole slide scanning platform for H&E stained sections and immunofluorescent images were collected on a Leica TCS SP8 2 photon laser scanning microscope.

TABLE 3
Antibodies used in Examples

| Antigen | | | | | | |
|---|---|---|---|---|---|---|
| Target | Conjugation | Clone | Catalogue # | Company | 1°/2° | Source |
| Cytokeratin-8 | AlexaFluor ®647 | EP1628Y | ab192468 | Abcam | 1° | rabbit monoclonal |
| Cytokeratin-14 | Biotin | LL002 | MA5-11596 | ThermoFisher | 1° | mouse IgG3 |
| EpCAM | none | EpCAM/TROP1 | AF960 | R&D System | 1° | goat polyclonal IgG |
| EYA1 | none | L-19 | Sc15094 | Santa Cruz Biotechnology | 1° | goat polyclonal IgG |
| FOXA2 (HNF-3beta) | none | M-20 | Sc-6554 | Santa Cruz Biotechnology | 1° | goat polyclonal IgG |
| HLA-DR | none | L243 | 307602 | Biolegend | 1° | mouse IgG2a kappa |
| HOXA3 | none | H-165 | Sc-28598 | Santa Cruz Biotechnology | 1° | rabbit polyclonal |
| ISL1/2 | none | 39.4D5 | 39.4D5 | Devl S Hybridoma | 1° | Mouse IgG3 |
| Keratin 5 (K5) | none | A-16 | Sc-17090 | Santa Cruz Biotechnology | 1° | goat polyclonal IgG |
| Keratin 8 (K8) | none | A-9 | Sc-374275 | Santa Cruz Biotechnology | 1° | mouse IgG2b kappa |
| OCT4 | none | C-10 | Sc-5279 | Santa Cruz Biotechnology | 1° | mouse IgG2b kappa |
| SIX1 | none | A-20 | Sc-9709 | Santa Cruz Biotechnology | 1° | goat polyclonal IgG |
| SOX2 | none | Y-17 | Sc-17320 | Santa Cruz Biotechnology | 1° | goat polyclonal IgG |
| TBX1 | none | TBX1 | 34-9800 | Invitrogen | 1° | rabbit polyclonal IgG |
| UEA-1 | biotin | N/A | B-1065 | Vector Laboratories | 1° | N/A |
| Donkey Anti- | AlexaFluor ®488 | N/A | A-11055 | ThermoFisher | 2° | donkey polyclonal IgG |
| goat IgG (H + L) | | | | | | |
| Donkey Anti- | AlexaFluor ®546 | N/A | A-11056 | ThermoFisher | 2° | donkey polyclonal IgG |
| goat IgG (H + L) | | | | | | |
| Donkey Anti- | AlexaFluor ®488 | N/A | A-21202 | ThermoFisher | 2° | donkey polyclonal IgG |
| mouse IgG (H+L) | | | | | | |
| Donkey Anti- | Cy ™3 | N/A | A-10036 | ThermoFisher | 2° | donkey polyclonal IgG |
| mouse IgG (H + L) | | | | | | |
| Donkey Anti- | AlexaFluor ®488 | N/A | A-21206 | ThermoFisher | 2° | donkey polyclonal IgG |
| rabbit IgG (H + L) | | | | | | |
| Rat Anti- | AlexaFluor ®488 | SB84a | Ab172324 | Abcam | 2° | rat monoclonal IgG1 |
| mouse IgG2a | | | | | | kappa |
| Streptavidin | AlexaFluor ®555 | N/A | S32355 | ThermoFisher | 2° | N/A |

### Animals and Human Tissues

NOD-scid IL2Rgammanu^{null} (NSG, stock 005557) mice were obtained from the Jackson Laboratory and bred and housed in microisolator cages in a Helicobacter- and Pasteurella pneumotropica-free SPF barrier. Human fetal thymus and liver tissues (gestational age 17 to 20 weeks) were obtained from Advanced Biosciences Resource. Fetal liver tissues were cut into small pieces and incubated at 37° C. in Medium 199 (Corning) supplemented with 0.01 mg/ml DNase I from bovine pancreas (Sigma), 2.5 mM HEPES, 4 ug/ml Gentamicin (Gibco) and 1 WU/ml Liberase™ (Roche) to create a single cell suspension. Cells were filtered through a 70 um mesh cell strainer into up to 100 ml of Medium 199 supplemented as listed above without Liberase. Human mononuclear cells were enriched by density gradient centrifugation by layering liver cell suspension over 15 ml Ficoll (Histopaque-1077 Sigma). Mononuclear cells were collected, washed, resuspended in MACS buffer and CD34+ cells enriched by magnetic-activated cell sorting (MACS) to purity of approximately 80% CD34+ according to the manufacturer's protocol (Miltenyi). CD34+ cells were frozen in aliquots in 10% DMSO (Sigma) in Human serum AB (GEMCell).

Three to five mm³ fragments of human pediatric thymus from patients undergoing cardiac surgery were cryopreserved in 10% DMSO in human AB serum. To generate primary thymic mesenchyme, thymic pieces were thawed, dissociated with Liberase™ digestion as described above and plated at approximately 2×10⁴ cells per cm² in DMEM media supplemented with 10% fetal calf serum (Gemini Bio-Products). Medium was changed 48 hours later to remove non-adherent cells and every 3-4 days up to 3 weeks until cells were confluent. Cells at passage 7-10 were used for experiments and identity of cells verified by flow cytometry (CD45-CD105+CD90+EpCAM−) (Siepe et al. 2009). Use of human tissues/cells was approved by the Columbia University Irving Medical Center (CUIMC) Institutional Review Board and all experiments were performed in accordance with approved protocols.

### Humanized Mice

Six to ten week old NSG mice were thymectomized as described (Khosravi-Maharlooei et al. 2020) and allowed to recover for at least 3 weeks. After recovery, animals were conditioned with 1.8 Gy total body irradiation (TBI) via X-rays. Cryopreserved fetal swine thymus (60-90 days gestation) was thawed in Medium 199 supplemented with DNAse, gentamicin and HEPES as above. Fetal swine fragments (1-2 mm³) were injected or not with 2×10⁵ hES-derived TEPs with a 28 gauge syringe and coated with 50% matrigel (Corning) in Medium 199. Four to 24 hours after TBI, 1-2×10⁶ hES-derived TEPs mixed with 1-2×10⁶ thymic mesenchymal cells, 1-2×10⁶ thymic mesenchymal cells alone, fetal swine thymus injected with hES-TEPs or fetal swine thymus alone were implanted beneath the kidney capsule and 2×10⁵ fetal human CD34+ cells were injected intravenously. Peripheral human immune reconstitution was assayed every 2-3 weeks post-grafting after full recovery as indicated. Blood was collected from the tail vein and immune populations enriched by density gradient centrifugation with Ficoll as described above. At the time of euthanasia, thymus, spleen and peripheral blood were collected for analysis. Thymic grafts were dissected from the mouse kidney and divided into two pieces. One thymic fragment was crushed to evolve thymocytes and remaining stromal components were digested with Liberase™ as described above to create a single cell suspension for flow cytometric analysis. The second thymic fragment was embedded in OCT. Spleen was crushed, filtered through 70 um nylon filter and red blood cells lysed with hypotonic lysis buffer (ACK Gibco). Peripheral blood from cardiac puncture was enriched for white blood cells by density gradient centrifugation over Ficoll. All animal experiments were performed under protocols approved by the Columbia University Institutional Animal Care and Use Committee.

### Flow Cytometry

Human immune reconstitution and differentiation efficiency of hES-TEP cultures were determined by multi-parametric flow cytometry. To assay human immune reconstitution, single cell suspensions prepared from thymus graft, tissue from the anterior mediastinum, spleen and peripheral blood were prepared as described above. Day 4.5 embryoid bodies from hES-TEP cultures were dissociated into single cells with 0.05% trypsin/EDTA. Cells were stained with fluorochrome-labeled monoclonal antibodies against mouse and human cell surface antigens (Table 4). Cells were acquired on an LSRII or Fortessa (BD Biosciences) and data analysis completed with FlowJo software (TreeStar, Ashland Oreg.).

TABLE 4
Monoclonal antibodies against mouse
and human cell surface antigens

| | | Species | | Cata- | |
|---|---|---|---|---|---|
| Antigen | | Speci- | | logue | |
| Target | Conjugation | ficity | Clone | # | Company |
| CD3 | PerCP-Cy5.5 | human | SP34-2 | 552852 | BD |
| | | | | | Pharmingen |
| CD4 | V500 | human | RPA-T4 | 560768 | BD |
| | | | | | Pharmingen |
| CD8 | Alexa- | human | RPA-T8 | 557945 | BD |
| | Fluor ®700 | | | | Pharmingen |
| CD14 | PE | human | HCD14 | 325606 | Biolegend |
| CD19 | APC | human | HIB19 | 302212 | Biolegend |
| CD31 | Brilliant Violet | human | WM59 | 562855 | BD |
| | 695 | | | | Pharmingen |
| CD45 | PE-FR594 | human | II30 | 562279 | BD |
| | | | | | Pharmingen |
| CD45 | APC-Cy7 | mouse | 30-F11 | 557659 | BD |
| | | | | | Pharmingen |
| CD45RA | FITC | human | HI100 | 555488 | BD |
| | | | | | Pharmingen |
| CD45RO | Brilliant Violet | human | UCHL1 | 304236 | Biolegend |
| | 711 | | | | |
| CD105 | PE-Cy7 | human | SN6 | 25-1057 | eBiosciences |
| CD184 | PE-Cy5 | human | 12G5 | 15-9999 | eBiosciences |
| (CXCR7) | | | | | |
| CD197 | Brilliant Violet | human | G430H7 | 353208 | Biolegend |
| (CCR7) | 421 | | | | |
| CD326 | biotin | human | 1B7 | 13-9326 | eBiosciences |
| (EpCAM) | | | | | |
| CD326 | Brilliant Violet | human | EBA-1 | 563182 | BD |
| (EpCAM) | 605 | | | | Pharmingen |
| HLA- | APC | human | G46-2.6 | 555555 | BD |
| ABC | | | | | Pharmingen |
| G46-2.6 | PE-Cy7 | mouse | TER-119 | 557853 | BD |
| (Erythro | | | | | Pharmingen |
| cells) | | | | | |
| strepta- | Alexa- | N/A | N/A | S21374 | Life |
| vidin | Fluor ®647 | | | | Technologies |

### Statistics

Statistical analysis and comparisons were performed with Graph-Pad Prism 7.0 (GraphPad Software). Values for individual mice are shown in bar graphs with the height of the bar depicting the average+standard error of the mean. For qPCR data, Ct values normalized to internal control β-actin were graphed and two-tailed ratio paired student t-test was used to compare relative gene expression. For multiple comparisons (more than two) from several experimental groups against a single control group, one-way ANOVA with Dunnett's test was used. Gene correlations were evaluated with Pearson Correlation Coefficient with p<0.05 considered significant, linear regression was also performed and r-square determined. Euthanasia due to teratoma growth was plotted on a Kaplan-Meyer plot and analyzed by Mantel Cox Log-rank test to determine p-value. Comparisons between groups of mice were made with the nonparametric Mann-Whitney U test. Effects between transplant groups were resolved by calculating a two-way analysis of variance (ANOVA). When the two-way ANOVA was significant (p<0.05), Bonferonni's multiple comparison test was run at individual time points. P<0.05 was considered significant.

### Example 2—Direct Differentiation of hESCs to 3rd PP-Biased Pharyngeal Endoderm

The thymus is derived from the pharyngeal endoderm (PE), the anterior-most part of the endoderm germ layer. Directed differentiation of TECs from ESCs requires sequential induction of definitive endoderm (DE), anterior foregut (AFE) and PE, followed by specification of the thymus domain of the third pharyngeal pouch (3^{rd} PP) (Gordon and Manley 2011) (FIGS. 1A and 2A). ESCs were differentiated to DE to AFE as described previously, using Activin A, and then Noggin plus SB431542 (NS) (Kubo et al. 2004; D'Amour et al. 2005; Green et al. 2011) (FIG. 1B). Flow cytometric analysis showed co-expression of endoderm markers EpCAM and CXCR4 in 98.3% of cells at day 4.5 from dissociated embryoid bodies (FIG. 1C). Dual BMP/TGF-β inhibition after induction of DE yielded AFE with high efficiency (>90%) (Soh et al. 2014). Consistently, day 9 immunofluorescent staining showed that the majority of the cells expressed FOXA2 (endoderm) and SOX2 (foregut), confirming efficient specification to AFE (FOXA2+SOX2+) (results not shown).

Next, differentiation of AFE toward thymic fate was focused on HOXA3, TBX1, PAX9, PAX1, SIX1 and EYA1 are genes involved in the development of PE and formation of the 3rd PP (Manley and Condie 2010). Hence their expression was used as read-outs at culture day 15.

In humans, HOXA3 is observed throughout the 3rd PP endoderm and surrounding mesenchyme, while TBX1 is expressed in the core mesenchyme of the 1st, 2nd and 3^{rd} pharyngeal arches (PA) and in the 3rd PP endoderm (Farley et al. 2013). In the PE the expression of these two genes only overlap in the 3^{rd} PP (Farley et al. 2013). Retinoic acid (RA), a factor essential for morphogenesis of PA (Kopinke et al. 2006) and PP (Wendling et al. 2000), has been correlated with the expression of Hoxa3 (Diman et al. 2011) while Fgf8 prevalence in the PP overlaps with Tbx1 at E10.5 in mice (Vitelli et al. 2002). To mimic the physiological 3rd PP endoderm development, simultaneous expression of TBX1 and HOXA3 was induced by combined RA and FGF8b stimulation of AFE cells in protocol #1 (FIG. 1B). To confirm the role of RA, the protocol was tested without it in protocol #2. Addition of RA was essential for HOXA3 expression (FIG. 1D, protocol #1 vs #2), consistent with the results shown by Parent et al. 2013.

FGF10, FGF7, CHIR (Wnt signaling activator) and BMP4 are also factors known to regulate the read-out genes (Parent et al. 2013; Sun et al. 2013; Soh et al. 2014; Su et al. 2015). The effect of substituting FGF8 with these cytokines individually was investigated in protocol #1. Not only did FGF8b+RA bring about the highest expression for most read-out genes, it was the only combination (FIG. 2B) that could drive TBX1 expression (FIGS. 2B and 2C). The addition of BMP4, CHIR, FGF7, and FGF10 to the protocol using FGF8b+RA did not improve the expression of any 3rd PP markers (not shown).

Despite the expression of most read-out genes, FOXN1, the master regulator for TEC differentiation (Romano et al. 2013), was barely detectable at culture day 15 (not shown), leaving room for improvement. In mice, Pax9 and Pax1 are expressed in the four PPs and become restricted to subpopulations of TECs postnatally (Wallin et al. 1996; Hetzer-Egger et al. 2002). Thus, besides being AFE markers, Pax1 and Pax9 are also TEC markers. Although the expression of PAX9 and PAX1 was statistically higher in protocols #1 and #2 than the negative control (liver, ‘hepatic conditions’ (Gouon-Evans et al. 2006)) (FIG. 1E), Shh was introduced at culture day 7.5 as a strategy for further upregulation of PAX9 and PAX1, as Shh induces the expression of Pax1 and Pax9 in ventral somites (Furumoto et al. 1999). Both Shh and its receptor, PTC1, are expressed in human TECs and have been reported to contribute to TEC differentiation (Saldana et al. 2016; Sacedon et al. 2003).

Since Shh enhances RA clearance (Probst et al. 2002), RA exposure was reduced and replaced with Shh at day 6.5 (FIG. 1B). This led to an increase in PAX9 that was significant (2.5-fold; p<0.0001) and also in PAX1 approaching significance (5-fold; p=0.053) (FIG. 1D protocol #1 vs #3). TBX1 expression was also increased significantly, consistent with the reports showing that Shh induces Tbx1 expression in PE (FIG. 1D) (Garg et al. 2001).

Next it was tested whether increasing the exposure to FGF8b, starting at culture day 4.5, would serve to bias AFE development towards PE and enhance the expression of the 3rd PP genes. An equivalent expression of 3rd PP markers was observed in both protocols #3 and #4, which led to continued efforts to optimize both in parallel, to explore their potential beyond day 15 of the differentiation (FIG. 1F).

### Example 3—Distalization of 3rd PP

Despite improved expression of 3rd PP markers with the addition of FGF8b during the anteriorization and/or culture with Shh, day 15 cultures showed low FOXN1 expression (results not shown). In mice, Bmp4 is co-expressed with FoxN1 in the ventral/posterior prospective thymus domain of the 3rd PP endoderm at E11.5 (Moore-Scott and Manley 2005; Bleul and Boehm 2005). It was therefore hypothesized that addition of BMP4 might lead to better expression of FOXN1. Thus, the day 15 cultures were exposed to BMP4 (FIG. 2A protocols #3b and #4b). However, addition of BMP4 failed to induce expression of FoxN1 assayed at culture day 22 and 30 for protocols #3b and #4b (results not shown). It was hypothesized that insufficient expression of PAX9, which is also expressed in TECs after thymus organ formation (Manley and Condie 2010; Hetzer-Egger et al. 2002), might be the cause of poor FOXN1 expression.

Next it was tested whether the addition of Noggin would increase PAX9 expression. Noggin is a BMP4 antagonist and/or inhibitor expressed throughout the mesenchyme of the 3rd PA at E9.5 in mice, immediately adjacent to the early 3rd PP endoderm (Patel et al. 2006). BMP4 expression begins at E10.5 in cells of the 3rd PP endoderm (Patel et al. 2006). It was hypothesized that Noggin may diffuse from the mesenchyme to the 3rd PP endodermal cells right before BMP4 signaling arises in this area. To mimic this event, BMP4 was substituted with Noggin from day 16 to day 22 in protocols #3c and #4c (FIG. 2A). PAX9 expression was significantly increased in both protocols with the addition of Noggin (FIG. 2D).

Five-fold greater levels of FOXN1 expression levels were observed in protocol #4c (FGF8b during anteriorization) as compared to protocol #3c (FIG. 2E). Thus, protocol #4c was further optimized. To confirm that the cells were producing FOXN1 after addition of BMP4, FOXN1 expression was compared at day 21 vs day 30 using protocol #4c. FIG. 2F shows that FOXN1 expression was significantly higher at day 30 than day 21, confirming that BMP4 exposure had the potential to enhance FOXN1 expression after day 21. In protocol #4c, FOXN1 levels at day 30 were 8 times higher than at day 15 (FIG. 2G).

Gene expression of TEC markers at culture day 30 as compared to whole human fetal thymus lysates is shown in FIG. 3A. Although the thymus stromal sample was diluted by the presence of thymocytes, protocol 4c achieved 76% of the expression of FOXN1 seen in thymic lysates. This was markedly higher than the levels reported by other groups doing the same comparison (Parent et al. 2013; Sun et al. 2013; Su et al. 2015). Furthermore, PAX9, PAX1, DLL4, ISL1, EYA1, SIX1, IL7, K5, K8 and AIRE mRNA was detectable at comparable or higher levels than fetal thymus. To establish the reproducibility of this protocol in other hESC lines, the human H9 ES cell line was treated with protocol #4c. The expression of TEC markers ISL1, FOXN1, K5, K8, DLL4, AIRE and IL7 (FIG. 3B) was demonstrable in H9 cells differentiated with this protocol.

Immunostaining of protocol #4c cultures at day 15 revealed colonies positive for PE markers TBX1, EYA, ISL1 and SIX, that also co-stained with 3rd PP marker EpCAM (results not shown). At culture day 30 these colonies remained positive for EpCAM, a general epithelial marker, K5 and UEA-1, which are associated with mTECs and K8, which is associated with cTECs (results not shown). A strong correlation between the expression levels of FOXN1 and GCM2, a parathyroid marker that is also found in the 3rd PP, was also found (FIG. 3C). This suggested the presence of cells destined to mature towards parathyroid progenitors despite BMP4 exposure, showing an incomplete distalization of the 3rd PP (Gordon et al. 2001). IL7 is an essential cytokine produced by TECs that promotes the survival, differentiation, and proliferation of thymocytes (Zamisch et al. 2005), as well as CD205, which functions as an endocytic receptor in cTECs (Shakib et al. 2009). It was found that IL7 and CD205 expression was correlated to that of FOXN1 (FIG. 3C).

### Example 4—Determining Functional Competence of hES-TEPs

hES-TEPs differentiated with protocol #4c were tested for their ability to support thymopoiesis from human hematopoietic stem cells grafted in a humanized mouse. Persistence of undifferentiated pluripotent cells in cultures is a major clinical translational barrier to use of ES and iPSC derivatives. Grafting experiments revealed the presence of pluripotent cells at the time of transplant resulting in rapid uncontrolled outgrowth of cells from the graft and teratoma formation (results not shown). Consistent with these results, OCT4, a marker for pluripotent cells, was detected in hES-TEP cultures at day 30 (FIG. 4C) (Pan et al. 2002). However, TEPs at culture day 30 showed co-expression of OCT4 in EpCAM+ cells (results not shown) and qPCR analysis showed a correlation between the expression levels of FOXN1 and OCT4 (FIG. 4B) suggesting OCT4 expression could be part of TEC differentiation program.

Survivin inhibitor YM155 has been reported to selectively eliminate pluripotent cells (Lee et al. 2013). Treatment with YM155 in the final 24 hours culture was tested to see if it was sufficient to eliminate pluripotent cells (FIG. 4A). OCT4 expression was significantly reduced with YM155 treatment (FIG. 4C). Engraftment of untreated day 15 hES-TEPs resulted in teratomas in all of animals by 11 weeks post-transplant (FIG. 4D). hES-TEPs cultured to 30 days with and without YM155 showed decreased teratoma formation compared to day 15 TEP grafted untreated controls, with only 3 of 15 animals developing teratomas in the group that received YM155-treated cells (results not shown).

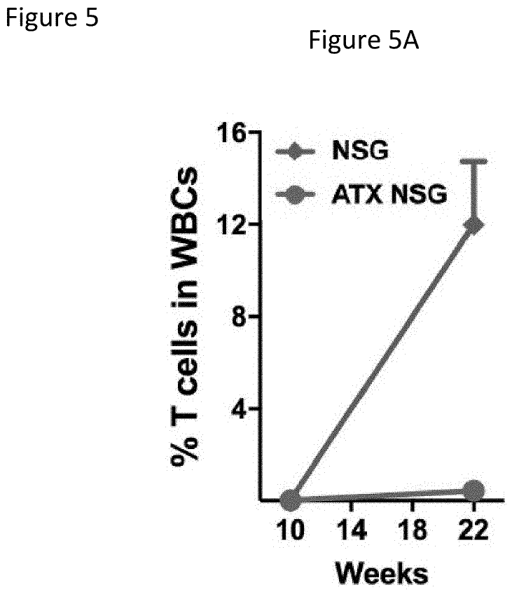

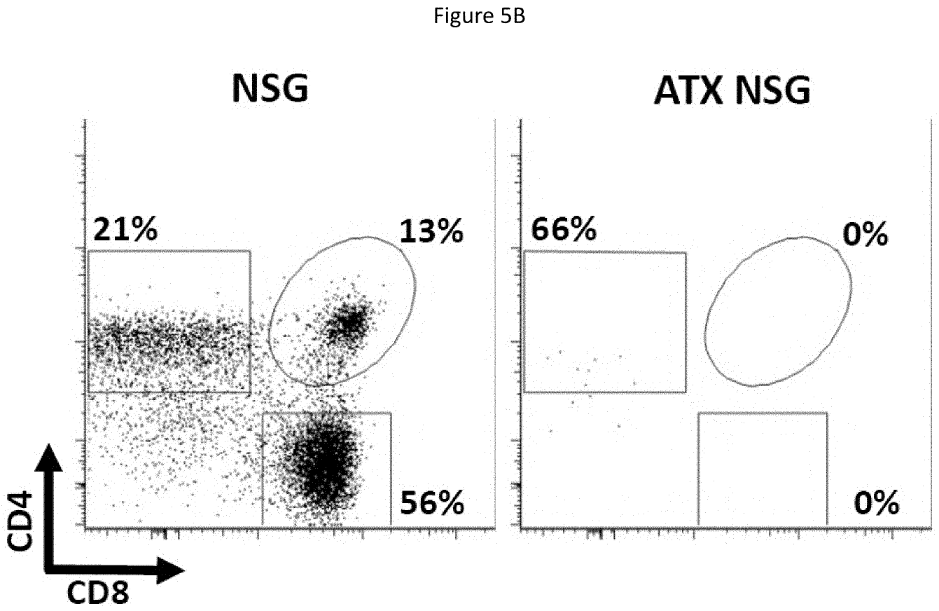

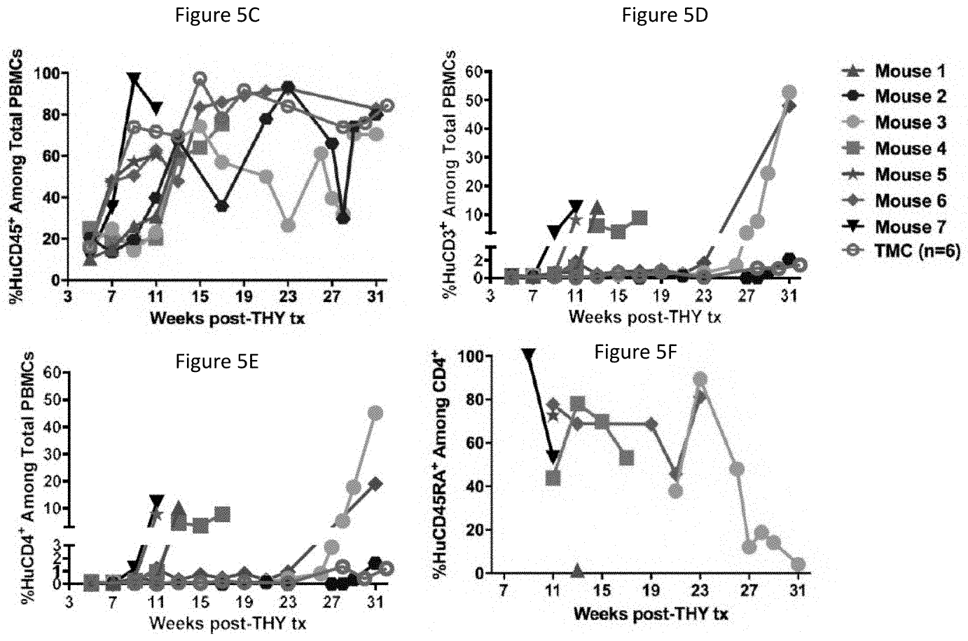

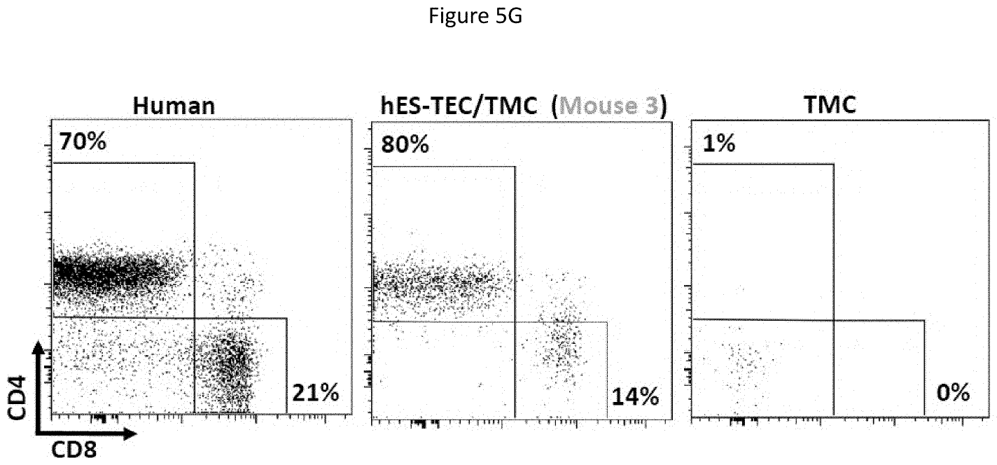

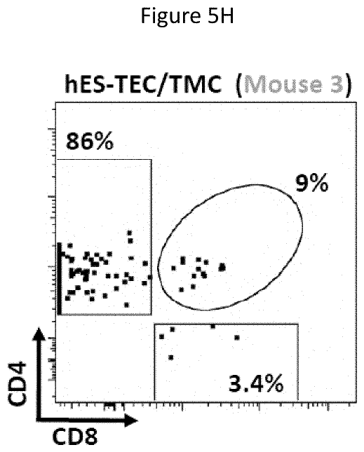

The native thymic rudiment of the NSG host was able to support low levels of thymopoiesis from human fetal liver-derived HSCs. A method to surgically remove both lobes of the native thymic rudiment from NSG mice was developed preventing T cell development in thymectomized (ATX) NSG animals grafted with human HSCs (Khosravi-Maharlooei et al. 2020). Complete removal of the native thymic rudiment in ATX mice was confirmed by collecting the connective tissue from the anterior mediastinum and assaying for the absence of CD4+CD8+ developing thymocytes (FIGS. 5A and 5B). Therefore, to assess the functional capacity of grafted hES-derived TEPs, all subsequent recipients were thymectomized.

### Example 5—Functional Thymic Organ Formation with hES-TEP/TMCs

To test the functional capacity of cultured hES-TEPs to support thymopoiesis, hES-TEP clusters (generated using protocol #4c) mixed with human thymic mesenchymal cells (TMCs), or TMCs alone, were grafted under the renal capsule of ATX NSG mice injected with i.v. 2×10⁵ human HSCs. Total human CD45+ cells in peripheral blood were shown for all mice, with human chimerism averaging 61%+21% among hES-TEP/TMC and 81%+13% among TMCs grafted mice from 11 to 31 weeks post-humanization (FIG. 5C). Human HSC engraftment resulted in dominant B cell production (data not shown). As early as 9 weeks post-TEP grafting under the renal capsule, human CD3+ T cells were detected at greater than 1% of total human blood cells in two mice grafted with hES-TEP/TMCs and were eventually detected in 6 of 7 hES-TEP-grafted mice, while TMC grafted controls did not show peripheral T cell reconstitution (FIG. 5D). T cells were skewed toward the CD4+ rather than the CD8+ lineage, however, 4 of 7 hES-TEP/TMC grafted mice developed both CD4+ and CD8+ cells (FIGS. 5E and 5G). CD4+ cells were further assayed for the expression of the naïve T cell marker CD45RA and the effector/memory T cell marker CD45RO. In the 4 mice that developed CD4+ and CD8+ T cells, CD4+ T cells had a predominantly naïve phenotype (CD45RA+CD45RO−), consistent with de novo thymopoiesis (FIG. 5F). Over time CD4+ T cells converted to an effector/memory phenotype (CD45RA−CD45RO+), consistent with arrest of thymopoiesis and lymphopenic expansion.

A low frequency of CD4+CD8+ double positive cells was present in the hES-TEP/TMC (FIG. 5H). hES-TEP/TMC grafts expanded slightly in volume and presented a disorganized architecture with no discernable cortical or medullary regions in hematoxylin and eosin stains (results not shown). In addition, cells from the hES-TEC/TMC graft appeared to penetrate the renal parenchyma, suggesting the presence of multiple cell types differentiating from TEP cultured cells in vivo. Despite disorganized architecture, some cells in the hES-TEC/TMC grafts co-tained with the TEC markers EpCAM, Pancytokeratin and human MHC II (HLA-DR), suggesting terminal differentiation and survival of the hES-TECs long-term (results not shown).

### Example 6—A Strategy for Testing the Impact of hES-TECs: Evidence for Integration into Porcine Thymus Grafts

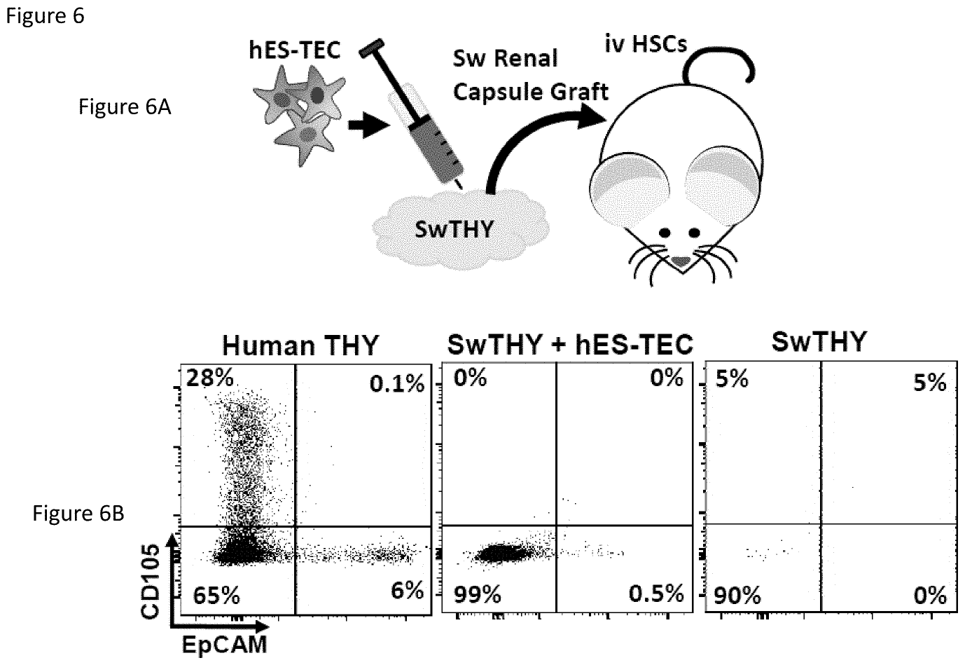

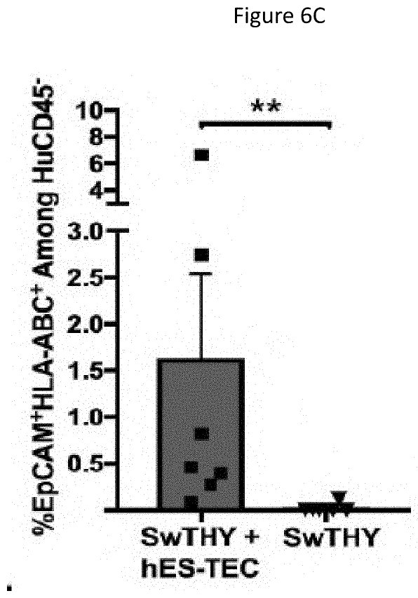

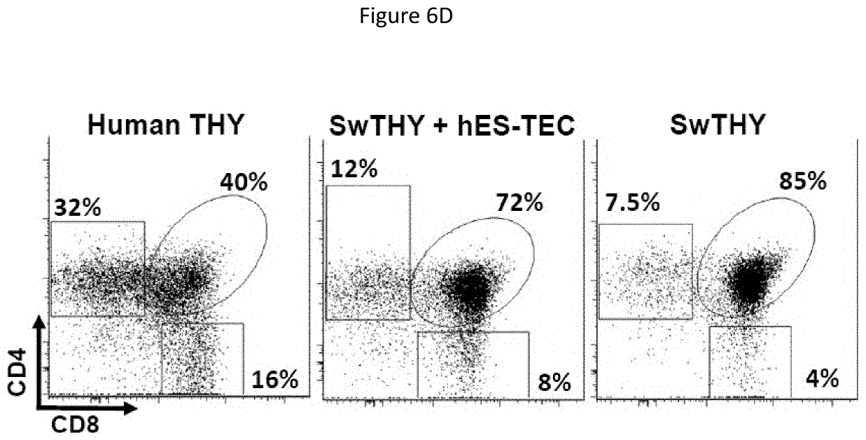

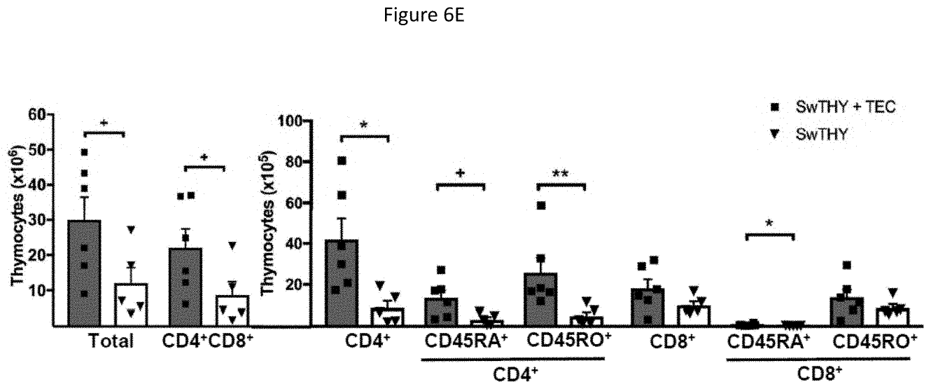

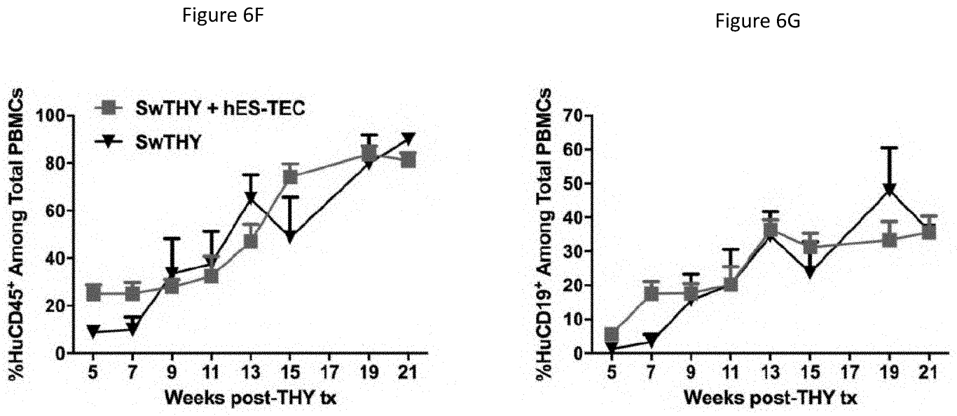

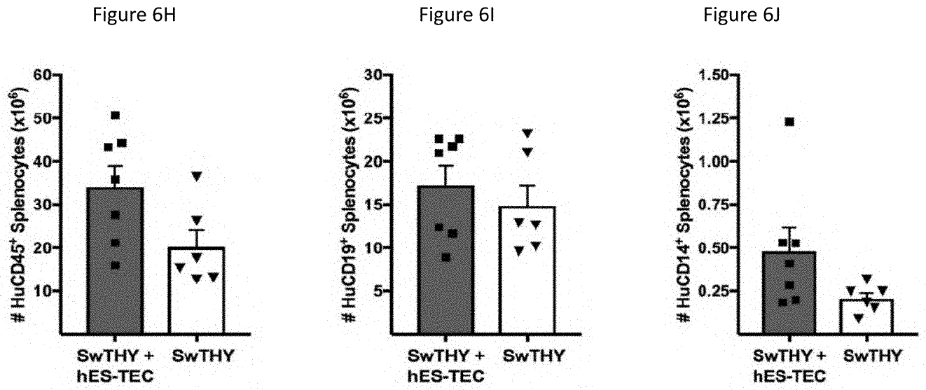

It was hypothesized that the ability of hES-TEPs to generate bona fide thymic tissue in vivo might be limited by the absence of a thymic structural scaffold or other cell types needed to generate a functioning thymus. To address this possibility, the survival and function of hES-TEPs (generated by protocol #4c) injected into a porcine fetal thymus graft in humanized mice was investigated. See FIG. 6A. Previously, it was shown that fetal swine thymus (SwTHY) supports robust thymopoiesis from human fetal liver-derived HSCs in NOD-scid or NSG mice (Kalcheuer et al. 2014; Nikolic and Sykes 1999; Nauman et al. 2019)

The presence of hES-TECs was analyzed by flow cytometry and immunofluorescence in injected SwTHY grafts 18-22 weeks post-transplant. Stromal cells from half of the thymus graft were dissociated with Liberase™ and stained for markers of human cells (huCD45 and HLA-ABC), thymic fibroblasts (CD105) and epithelial cells (EpCAM). Distribution of CD105 and EpCAM cells for SwTHY+hES-TEC and SwTHY are shown for huCD45− HLA-ABC+ cells (FIG. 6B). HuCD45-HLA-ABC+CD105-EpCAM+ were detected at a frequency of 1.6%+2.3% in the hES-TEC injected thymi, whereas they were undetectable in non-injected SwTHY, as expected (FIGS. 6B and 6C). Intact thymic grafts were stained with epithelial cell marker cytokeratin 14 and anti-human pan-MHCII (HLADR). Cytokeratin 14 is expressed on human and swine epithelial cells (red). HLA-DR is expressed on human antigen presenting cells seeding the thymic graft differentiated from human HSCs in the bone marrow and on terminally differentiated human TECs (green). Confocal microscopy showed colocalized HLA-DR and cytokeratins expressed by hES-TECs (yellow) in the injected SwTHY but not in uninjected SwTHY (results not shown). hES-TECs were detected in 6 of 7 SwTHY+hES-TEC thymic grafts.

### Example 7—hES-TEP Injection into Swine Thymus Improved Human Thymopoiesis

Thymocytes in the terminal stages of differentiation were assayed by flow cytometry to determine if hES-TECs supported improved human thymopoiesis. Distribution of single positive (SP) CD4+, CD8+ and double positive (DP) CD4+CD8+ cells in the SwTHY+hES-TEC and SwTHY grafts were similar to those in human pediatric thymus (FIG. 6D). hES-TECs in SwTHY led to a significant increase in the total number of thymocytes and CD4+CD8+DP cells compared to SwTHY grafts (FIG. 6E). The frequency and absolute number of CD4+SP, CD4+CD45RA+ and CD4+CD45RO+ developing T cells were significantly increased in SwTHY+hES-TEC compared to SwTHY grafts (FIG. 6E). These data suggested that hES-TECs may facilitate human thymopoiesis by providing human MHC interactions necessary for thymocyte survival from the double positive stage through terminal differentiation.

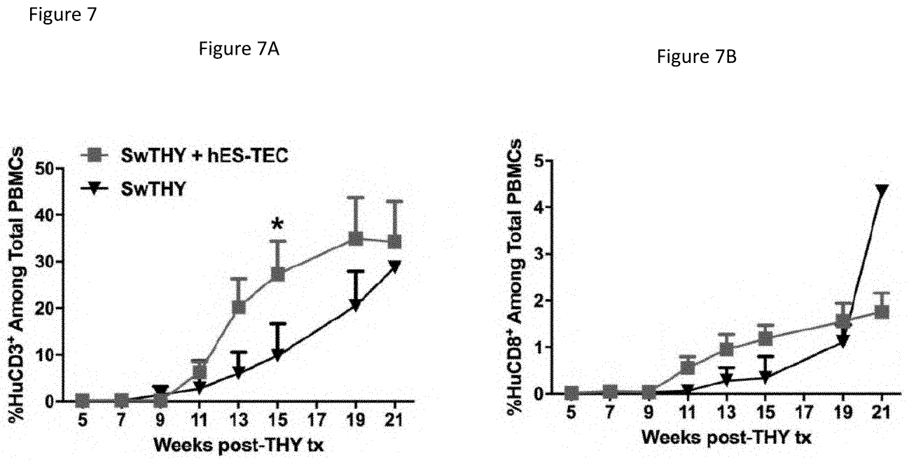

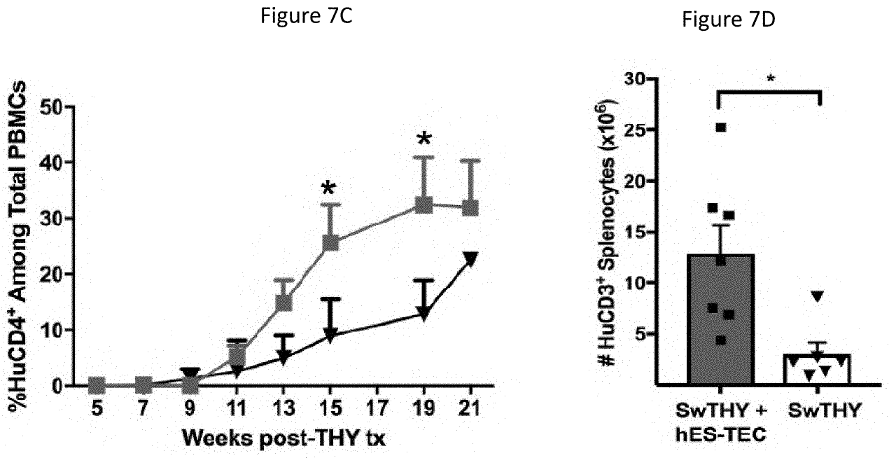

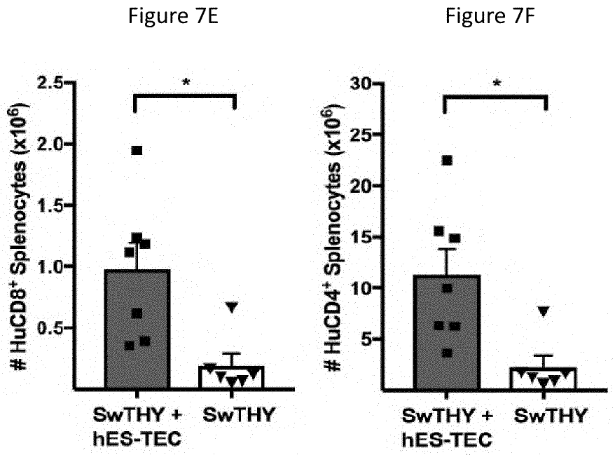

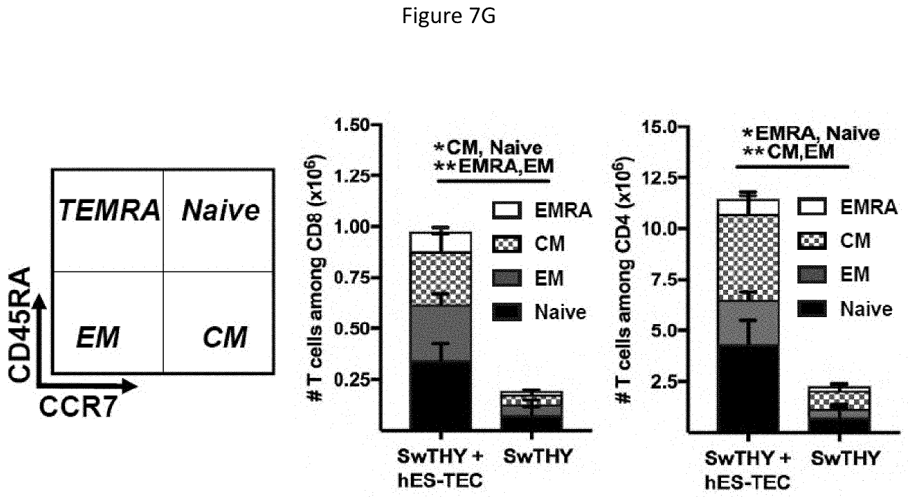

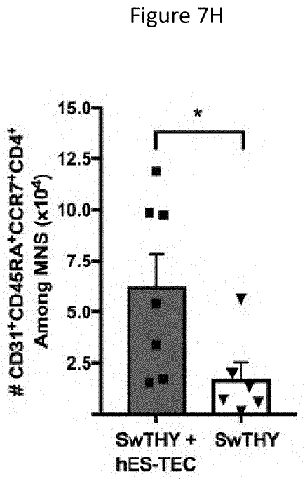

Next it was tested if the injection of hES-TEPs into SwTHY compared to uninjected SwTHY grafted under the renal capsule altered T cell frequency and phenotype in the periphery of HSC injected mice (FIG. 6A). Animals grafted with SwTHY or SwTHY injected with hES-TEPs (SwTHY+hES-TEC) developed robust human chimerism with a similar frequency of B cells, averaging approximately 30%+14% from 11 to 21 weeks post-humanization in peripheral blood (FIGS. 6F and 6G). Comparative kinetics of T cell reconstitution demonstrated a significant increase in the proportion of CD3+ T cells due to an increase in the frequency of CD4+ T cells in the blood of the SwTHY+hES-TEC group compared to the SwTHY group (FIG. 7A).

As a primary immune organ, splenic immune populations were assayed to determine if hES-TEC injection altered the frequency or absolute number cells. Frequencies and total numbers of human immune cells were comparable between SwTHY+hES-TEC and SwTHY groups (FIG. 6H). Similarly, there was no difference between groups in the number of CD19+ B cells and CD14+ monocytes (FIGS. 6I and 6J). Frequency and total number of CD3+ T cells was increased in the SwTHY+hES-TEC group compared to the SwTHY grafted animals (FIG. 7E). Both CD8+ cytotoxic and CD4+ helper T cells were elevated in percentage and absolute number in the SwTHY+hES-TEP injected group compared to SwTHY controls (FIG. 7F).

Phenotypic and functional subgroups of CD4 and CD8 T cells were defined based on expression of chemokine receptor CCR7 and CD45RA to delineate naïve (CD45RA+CCR7+), central memory (Tcm) (CD45RA-CCR7+), effector memory (Tem) (CD45RA-CCR7-) and terminally differentiated effector memory cells re-expressing CD45RA (TEMRA) (CD45RA+CCR7−) populations (FIG. 7G) (Thome et al. 2014). Consistent with an increase in the number of T cells in the SwTHY+hES-TEP grafted animals, naïve, Tcm, Tem and TEMRA were significantly increased in both the CD4+ and CD8+ T cell compartments (FIG. 7G). CD31 (platelet/endothelial cell adhesion molecule-1 or PECAM-1) is expressed by new naïve CD4+ T cells recently emigrating from the thymus. SwTHY+TEP injected animals showed a significant increase in the number of CD31+ cells among naïve CD4+ T cells compared to SwTHY controls (FIG. 7H), consistent with the interpretation that hES-TECs contribute to human T cell development.

### REFERENCES

Bleul and Boehm, BMP Signaling Is Required for Normal Thymus Development. The Journal of Immunology, 2005. 175(8): p. 5213.
Bredenkamp et al., An organized and functional thymus generated from FOXN1-reprogrammed fibroblasts. Nature cell biology, 2014. 16(9): p. 902-908.
D'Amour et al., Efficient differentiation of human embryonic stem cells to definitive endoderm. Nature Biotechnology, 2005. 23: p. 1534.
Diman et al., A Retinoic Acid Responsive Hoxa3 Transgene Expressed in Embryonic Pharyngeal Endoderm, Cardiac Neural Crest and a Subdomain of the Second Heart Field. PLoS ONE, 2011. 6(11): p. e27624.
Farley et al., Dynamics of thymus organogenesis and colonization in early human development. Development (Cambridge, England), 2013. 140(9): p. 2015-2026.
Furumoto et al., Notochord-Dependent Expression of MFH1 and PAX1 Cooperates to Maintain the Proliferation of Sclerotome Cells during the Vertebral Column Development. Developmental Biology, 1999. 210(1): p. 15-29.
Garg et al., Tbx1, a DiGeorge Syndrome Candidate Gene, Is Regulated by Sonic Hedgehog during Pharyngeal Arch Development. Developmental Biology, 2001. 235(1): p. 62-73.
Green et al., Generation of anterior foregut endoderm from human embryonic and induced pluripotent stem cells. Nature Biotechnology, 2011. 29: p. 267.
Gordon et al., Gcm2 and Foxn1 mark early parathyroid- and thymus-specific domains in the developing third pharyngeal pouch. Mechanisms of Development, 2001. 103(1): p. 141-143.
Gordon, Hox genes in the pharyngeal region: how Hoxa3 controls early embryonic development of the pharyngeal organs. Int J Dev Biol, 2018. 62(11-12): p. 775-783.
Gordon and Manley, Mechanisms of thymus organogenesis and morphogenesis. Development (Cambridge, England), 2011. 138(18): p. 3865-3878.
Gouon-Evans et al., BMP-4 is required for hepatic specification of mouse embryonic stem cell-derived definitive endoderm. Nature Biotechnology, 2006. 24(11): p. 1402-1411.
Hetzer-Egger et al., Thymopoiesis requires Pax9 function in thymic epithelial cells. European Journal of Immunology, 2002. 32(4): p. 1175-1181.
Huang et al., Efficient generation of lung and airway epithelial cells from human pluripotent stem cells. Nat Biotechnol, 2014. 32(1): p. 84-91.
Kischeuer et al., Xenograft tolerance and immune function of human T cells developing in pig thymus xenografts. J Immunol, 2014. 192(7): p. 3442-50.
Khosravi-Maharlooei et al., Rapid thymectomy of NSG mice to analyze the role of native and grafted thymi in humanized mice. Eur J Immunol, 2020. 50(1): p. 138-141.
Kopinke et al., Retinoic acid is required for endodermal pouch morphogenesis and not for pharyngeal endoderm specification. Developmental Dynamics, 2006. 235(10): p. 2695-2709.
Kubo et al., Development of definitive endoderm from embryonic stem cells in culture. Development, 2004. 131(7): p. 1651.
Lai and Jin, Generation of thymic epithelial cell progenitors by mouse embryonic stem cells. Stem Cells, 2009. 27(12): p. 3012-20.
Lee et al., Inhibition of pluripotent stem cell-derived teratoma formation by small molecules. Proceedings of the National Academy of Sciences of the United States of America, 2013. 110(35): p. E3281-E3290.
Manley and Condie, Transcriptional Regulation of Thymus Organogenesis and Thymic Epithelial Cell Differentiation, in Progress in Molecular Biology and Translational Science, A. Liston, Editor. 2010, Academic Press. p. 103-120.
Scott and Manley, Differential expression of Sonic hedgehog along the anterior-posterior axis regulates patterning of pharyngeal pouch endoderm and pharyngeal endoderm-derived organs. Developmental Biology, 2005. 278(2): p. 323-335.
Nauman et al., Reduced positive selection of a human TCR in a swine thymus using a humanized mouse model for xenotolerance induction. Xenotransplantation, 2019: p. e12558.
Nikolic and Sykes, Porcine thymus supports development of human T cells that are tolerant to porcine xenoantigens. Transplant Proc, 1999. 31(1-2): p. 924.
Pan et al., Stem cell pluripotency and transcription factor Oct4. Cell Research, 2002. 12(5): p. 321-329.
Parent et al., Generation of Functional Thymic Epithelium from Human Embryonic Stem Cells that Supports Host T Cell Development. Cell stem cell, 2013. 13(2): p. 10.1016/j.stem.2013.04.004.
Patel et al., Bmp4 and Noggin expression during early thymus and parathyroid organogenesis. Gene Expression Patterns, 2006. 6(8): p. 794-799.
Probst et al., SHH propagates distal limb bud development by enhancing CYP26B1-mediated retinoic acid clearance via AER-FGF signalling. Development (Cambridge, England), 2011. 138(10): p. 1913-1923.
Romano et al., FOXN1: A Master Regulator Gene of Thymic Epithelial Development Program. Frontiers in immunology, 2013. 4: p. 187-187.
Sacedón et al., Expression of Hedgehog Proteins in the Human Thymus. Journal of Histochemistry and Cytochemistry, 2003. 51(11): p. 1557-1566.
Saldana et al., Sonic Hedgehog regulates thymic epithelial cell differentiation. Journal of Autoimmunity, 2016. 68: p. 86-97.
Shakib et al., Checkpoints in the Development of Thymic Cortical Epithelial Cells. The Journal of Immunology, 2009. 182(1): p. 130.
Shimizu et al., Comparison of human T cell repertoire generated in xenogeneic porcine and human thymus grafts. Transplantation, 2008. 86(4): p. 601-10.
Siepe et al., Human neonatal thymus-derived mesenchymal stromal cells: characterization, differentiation, and immunomodulatory properties. Tissue Eng Part A, 2009. 15(7): p. 1787-96.
Soh et al., FOXN1(GFP/w) Reporter hESCs Enable Identification of Integrin-04, HLA-DR, and EpCAM as Markers of Human PSC-Derived FOXN1(+) Thymic Epithelial Progenitors. Stem Cell Reports, 2014. 2(6): p. 925-937.
Su et al., Efficient in vitro generation of functional thymic epithelial progenitors from human embryonic stem cells. Sci Rep, 2015. 5: p. 9882.
Sun et al., Directed Differentiation of Human Embryonic Stem Cells into Thymic Epithelial Progenitor-like Cells Reconstitutes the Thymic Microenvironment In Vivo. Cell Stem Cell, 2013. 13(2): p. 230-236.
Swann et al., Cooperative interaction of BMP signalling and Foxn1 gene dosage determines the size of the functionally active thymic epithelial compartment. Scientific reports, 2017. 7(1): p. 8492-8492.
Sykes and Sachs, Transplanting organs from pigs to humans. Sci Immunol, 2019. 4(41).
Tanabe et al., Role of Intrinsic (Graft) Versus Extrinsic (Host) Factors in the Growth of Transplanted Organs Following Allogeneic and Xenogeneic Transplantation. Am J Transplant, 2017. 17(7): p. 1778-1790.
Thome et al., Spatial map of human T cell compartmentalization and maintenance over decades of life. Cell, 2014. 159(4): p. 814-28.
Vitelli et al., A genetic link between Tbx1 and fibroblast growth factor signaling. Development, 2002. 129(19): p. 4605.
Vitelli et al., Partial Rescue of the Tbx1 mutant Heart Phenotype by Fgf8: genetic evidence of impaired tissue response to Fgf8. Journal of molecular and cellular cardiology, 2010. 49(5): p. 836-840.
Wallin et al., Pax1 is expressed during development of the thymus epithelium and is required for normal T-cell maturation. Development, 1996. 122(1): p. 23.
Wendling et al., Retinoid signaling is essential for patterning the endoderm of the third and fourth pharyngeal arches. Development, 2000. 127(8): p. 1553.
Yamada et al., Marked prolongation of porcine renal xenograft survival in baboons through the use of alpha1,3-galactosyltransferase gene-knockout donors and the cotransplantation of vascularized thymic tissue. Nat Med, 2005. 11(1): p. 32-4.
Zamisch et al., Ontogeny and Regulation of IL-7-Expressing Thymic Epithelial Cells. The Journal of Immunology, 2005. 174(1): p. 60.

1. A method of inducing differentiation of pluripotent stem cells into thymic epithelial cells (TECs) or thymic epithelial cell progenitors (TEPs) comprising the steps of
a. differentiating the pluripotent stem cells into definitive endoderm cells;
b. culturing the definitive endoderm cells and differentiating the definitive endoderm cells into anterior foregut cells by contacting or incubating the definitive endoderm cells with an agent which inhibits Bone Morphogenic Protein (BMP) and an agent which inhibits TGFβ signaling, and further contacting or incubating the definitive endoderm cells with an agent which stimulates the expression of HOXA3 and an agent which stimulates the expression of TBX1;
c. culturing the anterior foregut cells and differentiating the anterior foregut cells into pharyngeal endoderm cells by contacting or incubating the anterior foregut cells with an agent which stimulates the expression of HOXA3, an agent which stimulates the expression of TBX1 and an agent which stimulates the expression of PAX9 and PAX1;
d. culturing the pharyngeal endoderm cells and differentiating the pharyngeal endoderm cells into distal pharyngeal pouch (PP) specification cells, TECs or TEPs by contacting or incubating the pharyngeal endoderm cells with an agent which inhibits BMP; and
e. culturing the pharyngeal endoderm cells from step c. or step d. and differentiating the pharyngeal endoderm cells into distal pharyngeal pouch (PP) specification cells, TECs or TEPs by contacting or incubating the pharyngeal endoderm cells with BMP.

2. The method of claim 1, wherein step a. is performed for about one to about six days.

3. The method of claim 1, wherein in step a., the pluripotent stem cells are cultured in serum-free differentiation medium and contacted or incubated with BMP in an amount of about 0.5 ng/ml, human b Fibroblast Growth Factor (bFGF) in an amount of about 2.5 ng/ml and human Activin A in an amount of about 100 ng/ml.

4. The method of claim 1, wherein step b. is performed starting at about day 3 to about day 5 for about 2 days to about 3 days.

5. The method of claim 1, wherein in step b. the agent which inhibits BMP is selected from the group consisting of Noggin and dorsomorphin, the agent which inhibits TGF signaling is SB431542, the agent which stimulates the expression of HOXA3 is retinoic acid, and the agent which stimulate the expression of TBX1 is FGF8b.

6. The method of claim 1, wherein step c. is performed starting at about day 5 to about day 8 for about 6 days to about 10 days.

7. The method of claim 1, wherein in step c. the agent which stimulates the expression of HOXA3 is retinoic acid, the agent which stimulates expression of TBX1 is FGF8b, and the agent which stimulates the expression of PAX9 and PAX1 is Sonic Hedgehog (Shh).

8. The method of claim 7, wherein in step c. at about 24 hours FGF8b is used in an amount of about 50 ng/ml and retinoic acid is used in an amount of about 0.25 μM and at 48 hours FGF8b is used in an amount of about 50 ng/mL and Shh in an amount of about 100 ng/ml.

9. The method of claim 1, wherein step d. is performed starting at about day 12 to about day 18 for about 4 days to about 7 days.

10. The method of claim 1, wherein in step d. the agent which inhibits BMP is selected from the group consisting of Noggin and dorsomorphin.

11. The method of claim 1, wherein step e. is performed starting at about day 19 to about day 25 for about 5 days to about 15 days.

12. The method of claim 1, wherein in step e. BMP is used in an amount of about 50 ng/ml.

13. The method of claim 1, further comprising a step of contacting or incubating the TECs or TEPs at the end of the method with a survivin inhibitor, optionally wherein the survivin inhibitor is YM155.

14. (canceled)

15. A method for inducing differentiation of pluripotent stem cells into thymic epithelial cells (TECs), or thymic epithelial cell progenitors (TEPs) comprising the steps of:
a. differentiating the pluripotent stem cells into definitive endoderm cells by culturing the pluripotent stem cells in a serum-free differentiation medium and contacting or incubating the pluripotent stem cells with human Bone Morphogenic Protein (BMP), human basic Fibroblast Growth Factor (bFGF) and human Activin A;
b. differentiating the definitive endoderm cells from step a. into anterior foregut cells by culturing the definitive endoderm cells in the serum-free differentiation medium and contacting or incubating the definitive endoderm cells with Noggin, and SB431542, optionally further comprising retinoic acid or FGF8b;
c. differentiating the anterior foregut cells from step b. into pharyngeal endoderm cells by culturing the anterior foregut cells in the serum-free differentiation medium, and contacting or incubating the anterior foregut cells with FGF8b and retinoic acid followed by FGF8b and Sonic Hedgehog (Shh);
d. differentiating the pharyngeal endoderm cells from step c. into 3rd pharyngeal pouch specification cells, TECs or TEPs by culturing the pharyngeal endoderm cells in the serum-free differentiation medium and contacting or incubating the cells with Noggin; and
e. optionally further differentiating the pharyngeal endoderm cells from step c. or step d. into 3rd pharyngeal pouch specification cells, TECs or TEPs by culturing the pharyngeal endoderm cells in the serum-free differentiation medium and contacting or incubating the pharyngeal endoderm cells with BMP.

16. The method of claim 15, wherein the pluripotent stem cell is an embryonic stem cell or an induced pluripotent stem cell.

17. The method of claim 15, wherein step a. performed for about one to about six days.

18. The method of claim 15 wherein in step a., the BMP is used in an amount of about 0.5 ng/ml, the human b Fibroblast Growth Factor is used in an amount of about 2.5 ng/ml and the human Activin A is used in an amount of about 100 ng/ml.

19. The method of claim 15 wherein step b. is performed starting at about day 3 to about day 5 for about 2 days to about 3 days.

20. The method of claim 15, wherein in step b., the Noggin is used in an amount of about 200 ng/ml, the SB431542 is used in an amount of about 10 μM, the retinoic acid is used in an amount of about 0.25 μM, and the FGF8b is used in an amount of about 50 ng/mL.

21. The method of claim 15, wherein step c. is performed starting at about day 5 to about day 8 for about 6 days to about 10 days.

22. The method of claim 15, wherein in step c. at about 24 hours from the start of step c., FGF8b is used in an amount of about 50 ng/mL and Retinoic Acid is used in an amount of about 0.25 μM.

23. The method of claim 15, wherein in step c. at about 48 hours from the start of step c., FGF8b is used in an amount of about 50 ng/mL and Shh is used in an amount of about 100 ng/ml.

24. The method of claim 15, wherein step d. is performed starting at about day 12 to about day 18 for about 4 days to about 7 days.

25. The method of claim 15, wherein in step d. Noggin is used in an amount of about 100 ng/ml.

26. The method of claim 15, wherein step e. is performed starting at about day 19 to about day 25 for about 5 days to about 15 days.

27. The method of claim 15, wherein in step e. BMP is used in an amount of about 50 ng/ml.

28. The method of claim 15, further comprising a step of contacting or incubating the TECs or TEPs at the end of the method with a survivin inhibitor, optionally wherein the survivin inhibitor is YM155.

29. (canceled)

30. A thymic epithelial cell or a thymic epithelial cell progenitor obtained by the method comprising the steps of.
a. differentiating pluripotent stem cells into definitive endoderm cells by culturing the pluripotent stem cells in a serum-free differentiation medium and contacting or incubating the pluripotent stem cells with human Bone Morphogenic Protein (BMP), human basic Fibroblast Growth Factor (bFGF) and human Activin A;
b. differentiating the definitive endoderm cells from step a. into anterior foregut cells by culturing the definitive endoderm cells in the serum-free differentiation medium and contacting or incubating the definitive endoderm cells with Noggin, and SB431542, optionally further comprising retinoic acid or FGF8b;
c. differentiating the anterior foregut cells from step b. into pharyngeal endoderm cells by culturing the anterior foregut cells in the serum-free differentiation medium, and contacting or incubating the anterior foregut cells with FGF8b and retinoic acid followed by FGF8b and Sonic Hedgehog (Shh);
d. differentiating the pharyngeal endoderm cells from step c. into 3rd pharyngeal pouch specification cells, thymic epithelial cells, or thymic epithelial cell progenitor cells by culturing the pharyngeal endoderm cells in the serum-free differentiation medium and contacting or incubating the cells with Noggin; and
e. optionally, further differentiating the pharyngeal endoderm cells from step c. or step d. into 3rd pharyngeal pouch specification cells, thymic epithelial cells or thymic epithelial cell progenitor cells by culturing the pharyngeal endoderm cells in the serum-free differentiation medium and contacting or incubating the pharyngeal endoderm cells with BMP.

31. A method of preventing and/or treating a disease of the thymus, comprising administering to a subject in need thereof, a therapeutically effective amount of the thymic epithelial cell or thymic epithelial cell progenitor of claim 30, optionally wherein the disease is an autoimmune disease.

32. (canceled)

33. A method of recovering or restoring the impaired function of the thymus, comprising administering to a subject in need thereof, the thymic epithelial cell or thymic epithelial cell progenitor of claim 30, optionally wherein the impaired function of the thymus is due to injury, aging or congenital abnormality.

34.-46. (canceled)


SEQUENCE LISTING (34 sequences)

SEQ ID NO: 1 - DNA, 24 bp, Artificial Sequence
  Synthetic Primer
tttgaatgatgagccttcgtgccc

SEQ ID NO: 2 - DNA, 26 bp, Artificial Sequence
  Synthetic Primer
ggtctcaagtcagtgtacaggtaagc

SEQ ID NO: 3 - DNA, 20 bp, Artificial Sequence
  Synthetic Primer
caccacagatgtgtccgaga

SEQ ID NO: 4 - DNA, 18 bp, Artificial Sequence
  Synthetic Primer
agggctgaccgacgagat

SEQ ID NO: 5 - DNA, 24 bp, Artificial Sequence
  Synthetic Primer
acctctgcctttgtggtgaatgga

SEQ ID NO: 6 - DNA, 25 bp, Artificial Sequence
  Synthetic Primer
ggcagacacataacgctgtgctaaa

SEQ ID NO: 7 - DNA, 24 bp, Artificial Sequence
  Synthetic Primer
ttcaaccacccgttctccatcaac

SEQ ID NO: 8 - DNA, 26 bp, Artificial Sequence
  Synthetic Primer
ctgttcgtaggccttgaggtccattt

SEQ ID NO: 9 - DNA, 20 bp, Artificial Sequence
  Synthetic Primer
cggcacaacctatccctcaa

SEQ ID NO: 10 - DNA, 20 bp, Artificial Sequence
  Synthetic Primer
ttgtcgatcttggccggatt

SEQ ID NO: 11 - DNA, 24 bp, Artificial Sequence
  Synthetic Primer
agagttccacttcaaccgctacct

SEQ ID NO: 12 - DNA, 24 bp, Artificial Sequence
  Synthetic Primer
atgcccttgcccttctgatccttt

SEQ ID NO: 13 - DNA, 22 bp, Artificial Sequence
  Synthetic Primer
atgcacaacgagaggattttga

SEQ ID NO: 14 - DNA, 22 bp, Artificial Sequence
  Synthetic Primer
ctttgtgttcccaattccttcc

SEQ ID NO: 15 - DNA, 25 bp, Artificial Sequence
  Synthetic Primer
aaaccctccatgaactgtcctctcc

SEQ ID NO: 16 - DNA, 22 bp, Artificial Sequence
  Synthetic Primer
ccctgtgctccctactcctacc

SEQ ID NO: 17 - DNA, 25 bp, Artificial Sequence
  Synthetic Primer
ggaagccgtgacagaatgactacct

SEQ ID NO: 18 - DNA, 24 bp, Artificial Sequence
  Synthetic Primer
tggttatgttgctggacatgggtg

SEQ ID NO: 19 - DNA, 21 bp, Artificial Sequence
  Synthetic Primer
ctattctctcccgggcttaac

SEQ ID NO: 20 - DNA, 21 bp, Artificial Sequence
  Synthetic Primer
cagagagtcttggagctgatg

SEQ ID NO: 21 - DNA, 21 bp, Artificial Sequence
  Synthetic Primer
cccggctcctacgactattgc

SEQ ID NO: 22 - DNA, 24 bp, Artificial Sequence
  Synthetic Primer
ggaacgtattccttgcttgccctt

SEQ ID NO: 23 - DNA, 19 bp, Artificial Sequence
  Synthetic Primer
cctggatgcacttcttgga

SEQ ID NO: 24 - DNA, 19 bp, Artificial Sequence
  Synthetic Primer
cagagagctgtggccatgt

SEQ ID NO: 25 - DNA, 20 bp, Artificial Sequence
  Synthetic Primer
attgctggcacagtacagga

SEQ ID NO: 26 - DNA, 22 bp, Artificial Sequence
  Synthetic Primer
tggaattcatggacctccactt

SEQ ID NO: 27 - DNA, 20 bp, Artificial Sequence
  Synthetic Primer
gggcacctactgtgaactcc

SEQ ID NO: 28 - DNA, 20 bp, Artificial Sequence
  Synthetic Primer
gctgcccacaaagccataag

SEQ ID NO: 29 - DNA, 20 bp, Artificial Sequence
  Synthetic Primer
taccaggactcggcttctgt

SEQ ID NO: 30 - DNA, 20 bp, Artificial Sequence
  Synthetic Primer
atcatcgctgaggtcaaggc

SEQ ID NO: 31 - DNA, 20 bp, Artificial Sequence
  Synthetic Primer
tcctccactgatccttgttc

SEQ ID NO: 32 - DNA, 20 bp, Artificial Sequence
  Synthetic Primer
cttcaacttgcgagcagcac

SEQ ID NO: 33 - DNA, 24 bp, Artificial Sequence
  Synthetic Primer
ttgtacgggatcaaatgcgccaag

SEQ ID NO: 34 - DNA, 22 bp, Artificial Sequence
  Synthetic Primer
ccacacagcggaaacactcgat